(19) United States
(12) Patent Application Publication
(10) Pub. No.: US 2025/0288741 A1
(43) Pub. Date: Sep. 18, 2025

(54) INCREASED PARAMETER TITRATION FREQUENCY VIA ANALYTE HISTORY ADJUSTMENT

(71) Applicant: Abbott Diabetes Care Inc., Alameda, CA (US)

(72) Inventors: Erwin Satrya BUDIMAN, Fremont, CA (US); Yue RUAN, Oxford (GB); Roman HOVORKA, St. Albans (GB)

(21) Appl. No.: 19/080,496

(22) Filed: Mar. 14, 2025

Related U.S. Application Data
(60) Provisional application No. 63/565,368, filed on Mar. 14, 2024.

(51) Int. Cl. A61M 5/172; G16H 20/17

(52) U.S. Cl. CPC A61M 5/1723; G16H 20/17; A61M 2005/1726; A61M 2202/07; A61M 2230/201

(57) ABSTRACT

Disclosed herein are methods for more frequent titration of a parameter in the treatment of a disease in a patient. The disclosed methods negate the effect of past parameter doses on affected analyte history segments by adjusting the affected analyte history segment to compensate for the past parameter dose. The disclosed method allows for titration of a parameter without the need to reset analyte history segments every time a parameter is titrated, providing for the ability to titrate more frequently, and thus, improving the efficacy of patient treatment. This method can operate in conjunction with existing analyte monitoring systems (i.e., a CGM system).

### CROSS-REFERENCE TO RELATED APPLICATIONS

This application claims priority to U.S. provisional application 63/565,368, filed on Mar. 14, 2024, the entirety of which is hereby incorporated by reference.

### BACKGROUND

People with diabetes (PwD) must regularly test their glucose levels. Knowledge of their glucose levels is essential in helping a diabetic manage their diabetes. People with diabetes monitor their blood sugar before meals, at bedtime, and at various other points throughout a day.

Conventionally, a person with diabetes determines their glucose level by pricking their fingertip with a needle and testing the blood drop with a glucose meter. Depending on the glucose level, they may adjust their behavior, e.g., medication intake, dietary choices, and exercise regimen. They may also provide this data to doctors and medical professionals.

Continuous glucose monitoring (“CGM”) systems have been developed that allow PwD to monitor their glucose levels at regular intervals. CGM systems often rely on an under-the-skin, analyte sensor. An analyte is a substance whose chemical constituents are measured—in a CGM system, the analyte sensor measures the amount of glucose in body fluids. This obviates the need to draw blood with a needle and provides consistent, continuous readings. A coupled transmitter sends the readings to a wearable device, mobile device, or other external system. Glucose levels may be tracked every few seconds or minutes, 24 hours a day and 7 days a week.

A CGM system may analyze longer term trends. Exercise, diet, stress, and other life events may affect glucose levels. The data catalogued by a CGM system may lead to valuable insights. With this knowledge, a diabetic can determine their best diet and exercise regimen, adjust amounts of insulin and other medications, and develop an optimum plan to manage their diabetes. Various metrics have been developed to summarize a PwD's glycemic status based on the readings, e.g., the ambulatory glucose profile report (“AGP”), glucose management indicator (“GMI”), time-in-range (“TIR”), time-above-180 (“TA180”), and others.

Titration of insulin is typically necessary when adjusting doses. Glucose history recorded by a CGM system can be used as one of the primary inputs for the titration process. Of particular importance in dosing insulin is the patient's basal glucose level (e.g., the stable overnight concentration of glucose, the glucose history from midnight to 4 AM may be assigned as the segment most relevant to basal glucose). The glucose history data provided by a CGM system is segmented so that specific segments of time (e.g., overnight, breakfast, lunch, dinner) are associated with a particular insulin dose. Depending on how the segments are temporally defined, the datasets for different insulin doses may overlap in time.

Within the time overlap of the segments, a change in an insulin dose (e.g., unanticipated high or low dose) in a prior segment could affect the glucose history of a subsequent segment associated with another insulin dose. As a result, a past insulin titration can adversely affect the titration reliability of an affected insulin dose by altering affected glucose history segments. To prevent this undesired confluence, glucose history segments are reset whenever an insulin dose is administered. Resetting a glucose history segment means removing all glucose values that otherwise could be previously included or analyzed, and restarting the process of filling the glucose history segment with data whose timestamp is later than the reset time. Removed analyte values are not included in the rolling window of doses.

### BRIEF SUMMARY OF THE DISCLOSURE

According to some embodiments, the present invention aims to provide a method to adjust analyte history segments to negate the impact of prior parameter titration instances occurring within subsequent analyte history segments for the purposes of treating a disease.

In some embodiments, the method for frequent titration of a parameter for generating recommended dosage amounts includes the steps of storing analyte history segments where the plurality of analyte history segments may further include a first segment representing a first set of analyte values for a first time period and a second segment representing a second set of analyte values for a second time period. Another step includes storing parameter doses associated with the analyte history segments. The parameter doses may comprise a historic parameter dose. The method can further include performing an analysis of the analyte history segments to determine a hypothetical adjustment value for the analyte value in the analyte history segments.

In some embodiments, determining the hypothetical adjustment value may include additional steps of generating a difference value by subtracting the value of the historic parameter dose from a most recent parameter dose and setting the hypothetical adjustment value equal to a hypothetical change in analyte value caused by a hypothetical dose with the difference value.

Additional steps of the method can also include adjusting the stored analyte value to generate an adjusted stored patient analyte value based on the hypothetical adjustment value generating an updated parameter dose based on the adjusted stored patient analyte value. The adjusted stored patient analyte value may represent the hypothetical patient analyte value associated with a hypothetical administration of a parameter dose equivalent to the most recent parameter dose.

Disclosed herein are methods for more frequent titration of a parameter in the treatment of a disease in a patient. The disclosed methods may negate the effect of past parameter doses on affected analyte history segments by adjusting the affected analyte history segment to compensate for the past parameter dose. The disclosed method allows for titration of a parameter without the need to reset analyte history segments every time a parameter is titrated, providing for the ability to titrate more frequently, and thus, improving the efficacy of patient treatment. This method can operate in conjunction with existing analyte monitoring systems (i.e., a CGM system). Certain aspects of the disclosure have other steps or elements in addition to or in place of those mentioned above. The steps or elements will become apparent to those skilled in the art from a reading of the following detailed description when taken with reference to the accompanying drawings.

### BRIEF DESCRIPTION OF THE DRAWINGS

The accompanying drawings, which are incorporated herein and form a part of the specification, illustrate embodiments of the present disclosure and, together with the description, further serve to explain the principles of the disclosure and to enable a person skilled in the arts to make and use the embodiments.

FIG. 1A is a system overview of a sensor applicator, reader device, monitoring system, network, and remote system.

FIG. 1B is a diagram illustrating an operating environment of an example analyte monitoring system for use with the techniques described herein.

FIG. 2A is a block diagram depicting an example embodiment of a reader device.

FIG. 2B is a block diagram illustrating an example data receiving device for communicating with the sensor according to exemplary embodiments of the disclosed subject matter.

FIGS. 2C and 2D are block diagrams depicting example embodiments of sensor control devices.

FIG. 2E is a block diagram illustrating an example analyte sensor according to exemplary embodiments of the disclosed subject matter.

FIG. 3A is a proximal perspective view depicting an example embodiment of a user preparing a tray for an assembly.

FIG. 3B is a side view depicting an example embodiment of a user preparing a sensor applicator for an assembly.

FIG. 3C is a proximal perspective view depicting an example embodiment of a user inserting a sensor applicator into a tray during an assembly.

FIG. 3D is a proximal perspective view depicting an example embodiment of a user removing a sensor applicator from a tray during an assembly.

FIG. 3E is a proximal perspective view depicting an example embodiment of a patient applying a sensor using a sensor applicator.

FIG. 3F is a proximal perspective view depicting an example embodiment of a patient with an applied sensor and a used sensor applicator.

FIG. 4A is a side view depicting an example embodiment of a sensor applicator coupled with a cap.

FIG. 4B is a side perspective view depicting an example embodiment of a sensor applicator and cap decoupled.

FIG. 4C is a perspective view depicting an example embodiment of a distal end of a sensor applicator and electronics housing.

FIG. 4D is a top perspective view of an exemplary sensor applicator in accordance with the disclosed subject matter.

FIG. 4E is a bottom perspective view of the sensor applicator of FIG. 4D.

FIG. 4F is an exploded view of the sensor applicator of FIG. 4D.

FIG. 4G is a side cutaway view of the sensor applicator of FIG. 4D.

FIG. 5 is a diagram illustrating example operational states of the sensor according to exemplary embodiments of the disclosed subject matter.

FIG. 6 is a diagram illustrating an example operational and data flow for over-the-air programming of a sensor according to the disclosed subject matter.

FIG. 7 is a diagram illustrating an example data flow for secure exchange of data between two devices according to the disclosed subject matter.

FIG. 8 is a flowchart illustrating a method of generating an updated parameter dose based on adjusted analyte history segments and a method for generating the adjusted glucose history segments, according to some embodiments.

FIG. 9 is a flowchart illustrating a method of generating an updated insulin dose based on adjusted glucose history segments and a method for generating the adjusted glucose history segments, according to some embodiments.

FIG. 10A is a chart illustrating several glucose history segments, and the associated glucose concentrations and concentration of the basal and non-basal insulin doses, wherein the basal insulin doses are constant. S1 are segments relating to basal glucose, S2 are segments related to breakfast-related glucose, S3 are segments related to lunch-related glucose, and S4 are segments related to dinner-related glucose.

FIG. 10B is a chart illustrating several glucose history segments, and the associated glucose concentrations and concentration of the basal and non-basal insulin doses, wherein the basal insulin doses are non-constant. S1 are segments relating to basal glucose, S2 are segments related to breakfast-related glucose, S3 are segments related to lunch-related glucose, and S4 are segments related to dinner-related glucose. The upper dotted line is the hypothetical glucose history if all basal insulin doses were set at the 5 u dose. The lower dotted line is the hypothetical glucose history if all basal insulin doses were set at the 10 u dose.

FIG. 11 is a chart illustrating several glucose history segments, and the associated glucose and active insulin concentrations, and basal insulin doses, wherein all basal insulin dose concentrations are the same.

FIG. 12A is a chart illustrating several glucose history segments, and the associated glucose and active insulin concentrations, and basal insulin doses, wherein all basal insulin dose concentrations could differ from each other.

FIG. 12B is an equation for calculating the difference in value between a most recent basal insulin dose concentration and a historic basal insulin dose concentration (ΔD_{b}(d−i)).

FIG. 13A is a chart illustrating several glucose history segments, and the associated glucose concentrations, active insulin, basal insulin dose, and ΔD_{b}(d−i). The hypothetical change in active insulin caused by a basal insulin dose of ΔD_{b}(d−i) is displayed as a solid trapezoid on the “active insulin” row.

FIG. 13B is a chart illustrating several glucose history segments, and the associated glucose concentrations, active insulin, and basal insulin dose, and ΔD_{b}(d−i). The hypothetical change in active insulin caused by a basal insulin dose of 66 D_{b}(d−i) is displayed as a solid trapezoid on the “active insulin” row. The hypothetical glucose change caused by the hypothetical change in active insulin of FIG. 13A is displayed as a solid trapezoid on the “glucose concentration” row.

FIG. 13C is a chart illustrating several glucose history segments, and the associated glucose concentrations, and ΔD_{b}(d−i). The hypothetical glucose change caused by the hypothetical change in active insulin of FIG. 13A is displayed as a solid trapezoid on the “glucose concentration” row. A dotted line summing the individual hypothetical glucose changes together is shown on the “glucose concentration” row.

FIG. 13D is a chart illustrating several glucose history segments, and the associated glucose concentrations, and ΔD_{b}(d−i). The glucose concentrations are adjusted based on the dotted line of FIG. 13C producing an adjusted glucose history. The dotted line of FIG. 13C represents the hypothetical difference in glucose concentration caused by a hypothetical basal insulin dose equivalent to the difference between a most recent basal insulin dose and a historic basal insulin dose.

FIG. 14 is a chart illustrating several adjusted glucose history segments compared to several hypothetical glucose history segments where all basal dose are identical to the most recent dose.

FIG. 15 is a flowchart illustrating a method of generating a patient specific insulin sensitivity factor based on the median S1 glucose value for a glucose history segment and the difference in value between a most recent basal insulin dose concentration and a historic basal insulin dose concentration, according to some embodiments.

FIG. 16 is a chart correlating hypothetical median S1 glucose values for several glucose history segments and the difference in value between a most recent basal insulin dose concentration and historic basal insulin dose concentrations.

In the drawings, like reference numbers generally indicate identical or similar elements. Additionally, generally, the left-most digit(s) of a reference number identifies the drawing in which the reference number first appears.

### DETAILED DESCRIPTION

Reference will now be made in detail to representative embodiments illustrated in the accompanying drawings. Provided herein are methods for more frequent titration of a parameter in the treatment of a disease in a patient. If each glucose history segment in a patient's analyte history can be adjusted such that the impact of prior insulin titration instances occurring within subsequent glucose history segment can be negated, then more frequent titration can be achieved because glucose history segment resets are unnecessary.

The disclosed methods improve upon prior titration methods by implementing a procedure for negating the effect of past parameter changes on affected analyte history segments by adjusting the affected analyte history segment to compensate for the difference between a most recent parameter dose and the past parameter dose. The disclosed method allows for titration of a parameter without the need to reset analyte history segments every time that parameter is titrated, providing for the ability to titrate more frequently, and thus, improving the efficacy of patient treatment. In some embodiments, this method can be implemented in conjunction with existing analyte monitoring systems (i.e., a CGM system). In some embodiments, the disease is diabetes. In some embodiments, the analyte is glucose. In some embodiments, the parameter is an insulin dosing parameter such as long-acting insulin dose, the fixed dose element of mealtime insulin dose, specific mealtime fixed dose element such as fixed breakfast insulin dose, pre-meal insulin sensitivity factor, specific mealtime pre-meal insulin sensitivity parameter such as lunch pre-meal insulin sensitivity factor, meal carbohydrate-to-insulin ratio, specific meal carbohydrate-to-insulin ratio such as dinner carbohydrate-to-insulin ratio, post-meal insulin sensitivity factor, and specific meal insulin sensitivity factor such as lunch post-meal insulin sensitivity factor. In some embodiments, the insulin can be one or more of slow acting, fast acting, or pre-mixed insulin (i.e., a combination of fast-and slow acting insulin).

In some embodiments, glucose history half an hour prior and up to the start of breakfast plus glucose history from the peak glucose of the breakfast up to 4 hours since then or half an hour prior to the start of lunch, whichever occurs sooner, may be assigned as the segment most relevant to breakfast pre-meal insulin correction factor.

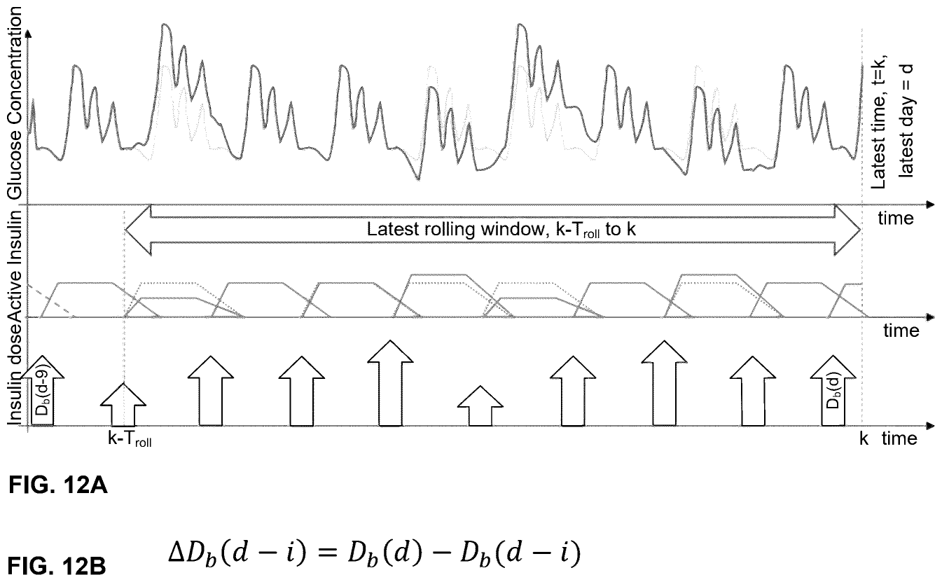

Resetting an analyte history segment means removing all analyte values that otherwise should be previously included or analyzed, and restarting the process of filling the analyte history segment with data whose timestamp is later than the reset time. Removed analyte values are not included in the rolling window of doses, for example, the rolling window shown in FIG. 12A.

Additionally provided herein are methods for generating a patient specific parameter sensitivity factor based on the median S1 analyte value for an analyte history segment and the difference in value between a most recent parameter dose concentration and a historic parameter dose concentration. S1 analyte value is the stable overnight concentration of analyte, the analyte history from midnight to 4 AM may be assigned as the segment most relevant to basal (S1) analyte. This method can provide for parameter doses that are more tailored to a particular patient, and thus, improve the efficacy of patient treatment.

The disclosed methods may be implemented as part of a software application installed on a mobile device in communication with a glucose monitoring device. The software application may be configured to implement the titration methods described herein. The software application may be configured with a graphical user interface for communicating status and results when executing the titration methods. This could also result in the improvement of the device that performs titration, e.g., an insulin delivery device. An example of such a device is an insulin pump.

The disclosed methods apply to slow acting, fast acting, or pre-mixed insulin (i.e., a combination of fast-and slow acting insulin).

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

As used herein “basal analyte” (i.e., where the analyte is glucose) refers the stable overnight concentration of an analyte, the analyte history from midnight to 4 AM may be assigned as the segment most relevant to basal (S1) analyte value. As used herein “basal parameter”/“basal parameter dose” (i.e., where the parameter is insulin) refers to a parameter dose associated with a “basal analyte” history segment. As used herein “non-basal parameter”/“non-basal parameter dose” (i.e., where the parameter is insulin) refers to a parameter dose not associated with a “basal analyte” history segment.

As used herein “Insulin Sensitivity Factor”, “Insulin Sensitivity” and “Insulin Correction Factor” are used interchangeably.

As used herein “titration” can refer to a value which is assumed to be relatively constant over a sufficiently long duration (e.g., half a year or more), or a value which is assumed to change over time.

As used herein “parameter” can refer to something whose optimal value is assumed to be constant or very slowly changing over time (i.e., insulin sensitivity (Si), post-meal glucose target (Gt), parameter dose, and insulin-to-carb ratio (ICR)).

As used herein “variable” can refer to something whose optimal value is assumed to be non-constant (i.e., pre-meal glucose value (which varies at every start of meal), pre-meal insulin dose, and meal carb amount (which varies depending on the meal choice at the time)).

Variables and parameters may both be used when determining the concentration and amount of a dose to be administered to a patient. For example, a pre-meal insulin dose is a variable amount and is a function of both parameters and variables. Parameters involved include, but are not limited to, insulin sensitivity (Si), post-meal glucose target (Gt), and insulin-to-carb ratio (ICR). Variables involved include, but are not limited to, pre-meal glucose value and meal carb amount.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Generally, embodiments of the present disclosure include methods for use with in vivo analyte monitoring systems. Furthermore, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

Furthermore, for each and every embodiment of a method disclosed herein, systems and devices capable of performing each of those embodiments are covered within the scope of the present disclosure. For example, embodiments of sensor control devices are disclosed and these devices can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps. These sensor control device embodiments can be used and can be capable of use to implement those steps performed by a sensor control device from any and all of the methods described herein.

Furthermore, the systems and methods presented herein can be used for operations of a sensor used in an analyte monitoring system, such as but not limited to wellness, fitness, dietary, research, information or any purposes involving analyte sensing over time. As used herein, “analyte sensor” or “sensor” can refer to any device capable of receiving sensor information from a user, including for purpose of illustration but not limited to, body temperature sensors, blood pressure sensors, pulse or heart-rate sensors, glucose level sensors, analyte sensors, physical activity sensors, body movement sensors, or any other sensors for collecting physical or biological information. Analytes measured by the analyte sensors can include, by way of example and not limitation, glucose, ketones, lactate, oxygen, hemoglobin A1C, albumin, alcohol, alkaline phosphatase, alanine transaminase, aspartate aminotransferase, bilirubin, blood urea nitrogen, calcium, carbon dioxide, chloride, creatinine, hematocrit, lactate, magnesium, oxygen, pH, phosphorus, potassium, sodium, total protein, uric acid, etc.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. CGM systems, for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (“NFC”), Bluetooth wireless communication, Radio Frequency Identification (“RFID”) protocol, or any other Radio Frequency (“RF”) method protocol. In vivo analyte monitoring systems can also operate without the need for finger stick calibration.

In vivo analyte monitoring systems can be differentiated from in vitro systems that contact a biological sample outside of the body (or ex vivo) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Exemplary In Vivo Analyte Monitoring System

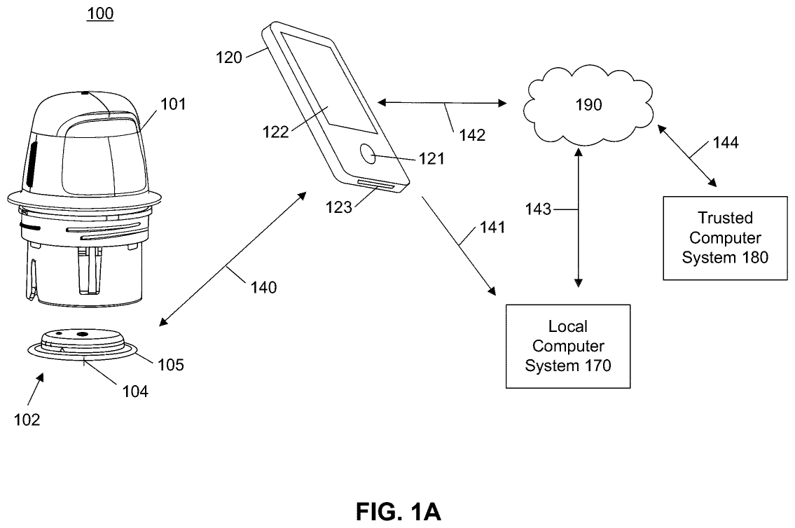

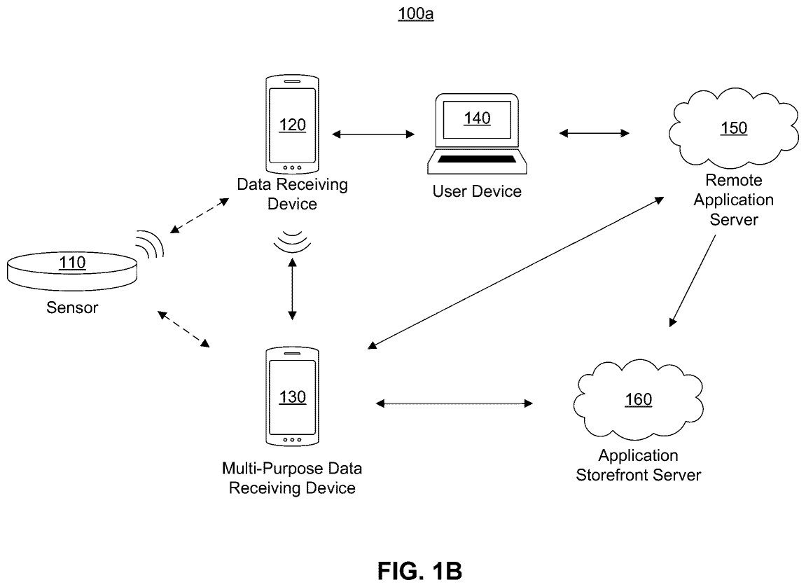

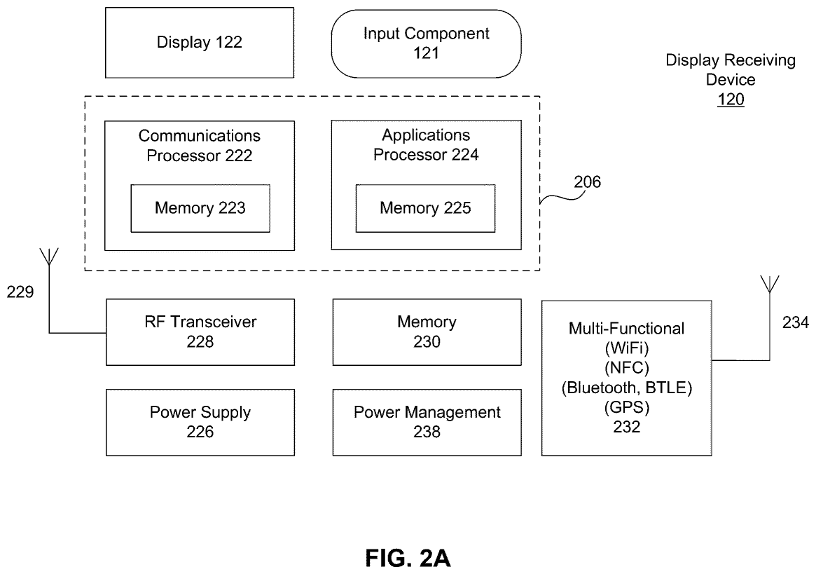

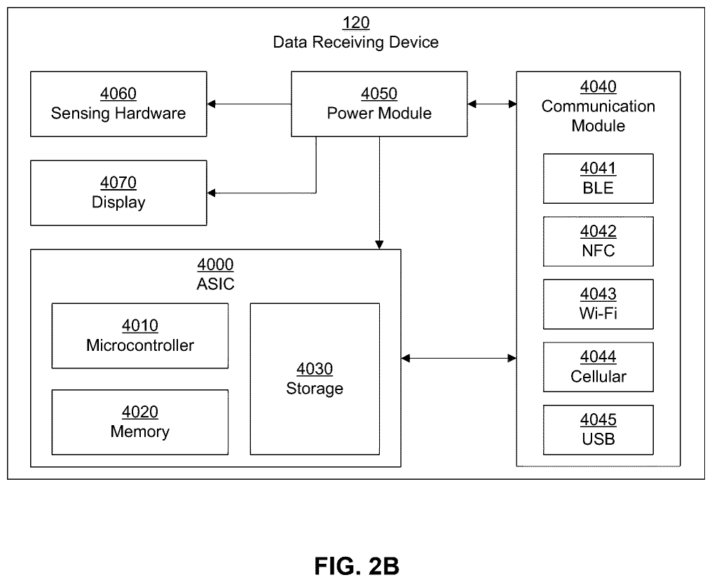

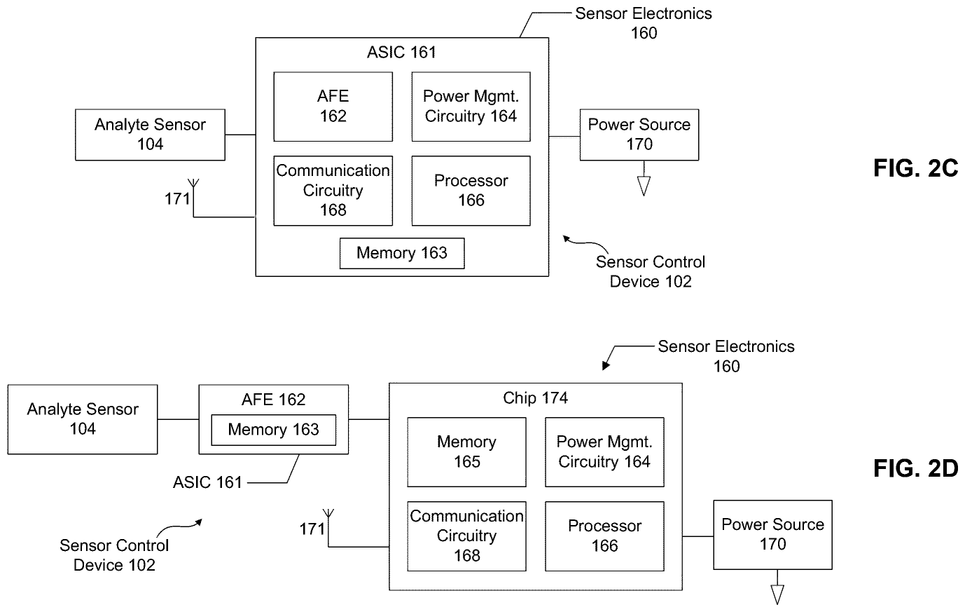

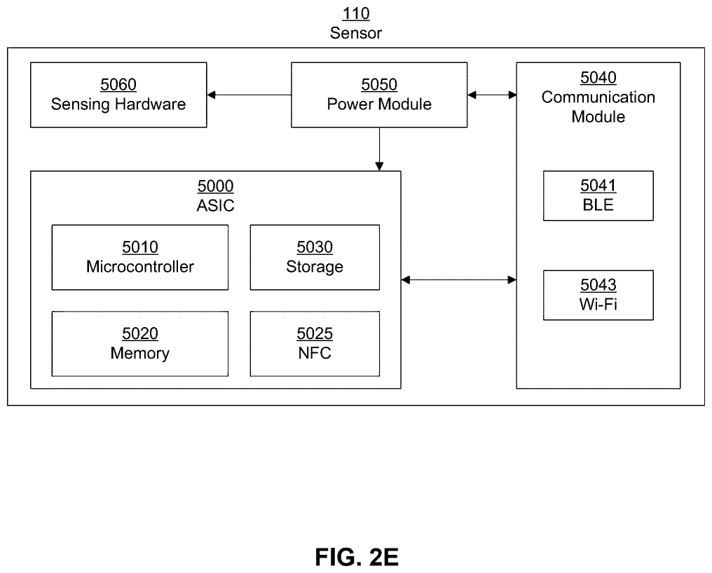

FIG. 1A is a conceptual diagram depicting an example embodiment of an analyte monitoring system 100 that includes sensor applicator 101, sensor control device 102, and data receiving device 120. Here, sensor applicator 101 can be used to deliver sensor control device 102 to a monitoring location on a user's skin where in vivo analyte sensor 104 is maintained in position for a period of time by adhesive patch 105. Sensor control device 102 is further described in FIGS. 2B and 2C, and can communicate with data receiving device 120 via a communication path 141 using a wired or wireless technique. Example wireless protocols include Bluetooth, Bluetooth Low Energy (“BLE” or “BTLE”), Bluetooth SMART, NFC, and others. Users can monitor applications installed in memory on data receiving device 120 using display 122 and input component 121 and the device battery can be recharged using power port 123. More detail about data receiving device 120 is set forth with respect to FIG. 2A below. Data receiving device 120 can communicate with local computer system 175 via a communication path 141 using a wired or wireless technique. Local computer system 175 can include one or more of a laptop, desktop, tablet, phablet, smartphone, set-top box, video game console, or other computing device and wireless communication can include any of a number of applicable wireless networking protocols including Bluetooth, BTLE, Wi-Fi or others. Local computer system 175 can communicate via communications path 143 with a network 190 similar to how data receiving device 120 can communicate via a communications path 142 with network 190, by wired or wireless technique as described previously. Network 190 can be any of a number of networks, such as private networks and public networks, local area or wide area networks, and so forth. A trusted computer system 180 can include a server and can provide authentication services and secured data storage and can communicate via communications path 144 with network 190 by wired or wireless technique.

FIG. 1B illustrates an operating environment of analyte monitoring system 100a capable of embodying the techniques described herein. Analyte monitoring system 100a may include a system of components designed to provide monitoring of parameters, such as analyte levels, of a human or animal body or can provide for other operations based on the configurations of the various components. As embodied herein, the system may include analyte sensor 110, or simply “sensor” worn by the user or attached to the body for which information is being collected. As embodied herein, analyte sensor 110 can be a sealed, disposable device with a predetermined active use lifetime (e.g., 1 day, 14 days, 30 days, etc.). Analyte sensor 110 may be applied to the skin of the user body and remain adhered over the duration of the sensor lifetime or can be designed to be selectively removed and remain functional when reapplied. Analyte monitoring system 100a can further include data receiving device 120 or multi-purpose data receiving device 130 configured as described herein to facilitate retrieval and delivery of data, including analyte data, from analyte sensor 110.

As embodied herein, analyte monitoring system 100a can include a software or firmware library or application provided, for example, via remote application server 150 or application storefront server 160, to a third-party and incorporated into multi-purpose data receiving device 130 such as a mobile phone, tablet, personal computing device, or other similar computing device capable of communicating with analyte sensor 110 over a communication link. Multi-purpose hardware can further include embedded devices, including, but not limited to insulin pumps or insulin pens, having an embedded library configured to communicate with analyte sensor 110. Although the illustrated embodiments of analyte monitoring system 100a include only one of each of the illustrated devices, this disclosure contemplates analyte monitoring system 100a incorporating multiples of each component interacting throughout the system. For example, and without limitation, as embodied herein, data receiving device 120 and/or multi-purpose data receiving device 130 can include multiples of each. As embodied herein, multi-purpose data receiving device 130 can communicate directly with analyte sensor 110 as described herein. Additionally or alternatively, multi-purpose data receiving device 130 can communicate with multi-purpose data receiving device 130 to provide analyte data, or visualization or analysis of the data, for secondary display to the user or other authorized parties.

### Exemplary Over-the-Air Updates

FIG. 1B further illustrates an example operating environment for providing over-the-air (“OTA”) updates for use with the techniques described herein. An operator of analyte monitoring system 100a can bundle updates for data receiving device 120 or analyte sensor 110 into updates for an application executing on multi-purpose data receiving device 130. Using available communication channels between data receiving device 120, multi-purpose data receiving device 130, and analyte sensor 110, multi-purpose data receiving device 130 can receive regular updates for data receiving device 120 or analyte sensor 110 and initiate installation of the updates on data receiving device 120 or analyte sensor 110. Multi-purpose data receiving device 130 acts as an installation or update platform for data receiving device 120 or analyte sensor 110 because the application that enables the multi-purpose data receiving device 130 to communicate with analyte sensor 110, data receiving device 120 and/or remote application server 150 can update software or firmware on data receiving device 120 or analyte sensor 110 without wide-area networking capabilities.

As embodied herein, remote application server 150 operated by the manufacturer of analyte sensor 110 and/or the operator of analyte monitoring system 100 can provide software and firmware updates to the devices of analyte monitoring system 100. In particular embodiments, remote application server 150 can provides the updated software and firmware to user device 140 or directly to a multi-purpose data receiving device. As embodied herein, remote application server 150 can also provide application software updates to application storefront server 160 using interfaces provided by the application storefront. Multi-purpose data receiving device 130 can contact the application storefront server 160 periodically to download and install the updates.

After multi-purpose data receiving device 130 downloads an application update including a firmware or software update for data receiving device 120 or analyte sensor 110, data receiving device 120 or analyte sensor 110 and multi-purpose data receiving device 130 establish a connection. Multi-purpose data receiving device 130 determines that a firmware or software update is available for data receiving device 120 or analyte sensor 110. Multi-purpose data receiving device 130 can prepare the software or firmware update for delivery to data receiving device 120 or analyte sensor 110. As an example, multi-purpose data receiving device 130 can compress or segment the data associated with the software or firmware update, can encrypt or decrypt the firmware or software update, or can perform an integrity check of the firmware or software update. Multi-purpose data receiving device 130 sends the data for the firmware or software update to data receiving device 120 or analyte sensor 110. Multi-purpose data receiving device 130 can also send a command to data receiving device 120 or analyte sensor 110 to initiate the update. Additionally or alternatively, multi-purpose data receiving device 130 can provide a notification to the user of multi-purpose data receiving device 130 and include instructions for facilitating the update, such as instructions to keep data receiving device 120 and multi-purpose data receiving device 130 connected to a power source and in close proximity until the update is complete.

Data receiving device 120 or analyte sensor 110 receives the data for the update and the command to initiate the update from multi-purpose data receiving device 130. Data receiving device 120 can then install the firmware or software update. To install the update, data receiving device 120 or analyte sensor 110 can place or restart itself in a so-called “safe” mode with limited operational capabilities. Once the update is completed, data receiving device 120 or analyte sensor 110 re-enters or resets into a standard operational mode. Data receiving device 120 or analyte sensor 110 can perform one or more self-tests to determine that the firmware or software update was installed successfully. Multi-purpose data receiving device 130 can receive the notification of the successful update. Multi-purpose data receiving device 130 can then report a confirmation of the successful update to remote application server 150.

In particular embodiments, storage memory 5030 of analyte sensor 110 in FIG. 2E includes OTP memory. The term OTP memory can refer to memory that includes access restrictions and security to facilitate writing to particular addresses or segments in the memory a predetermined number of times. Memory 5030 can be prearranged into multiple pre-allocated memory blocks or containers. The containers are pre-allocated into a fixed size. If storage memory 5030 is OTP memory, the containers can be considered to be in a non-programmable state. Additional containers which have not yet been written to can be placed into a programmable or writable state. Containerizing storage memory 5030 in this fashion can improve the transportability of code and data to be written to storage memory 5030. Updating the software of a device (e.g., the sensor device described herein) stored in an OTP memory can be performed by superseding only the code in a particular previously-written container or containers with updated code written to a new container or containers, rather than replacing the entire code in the memory. In a second embodiment, the memory is not prearranged. Instead, the space allocated for data is dynamically allocated or determined as needed. Incremental updates can be issued, as containers of varying sizes can be defined where updates are anticipated.

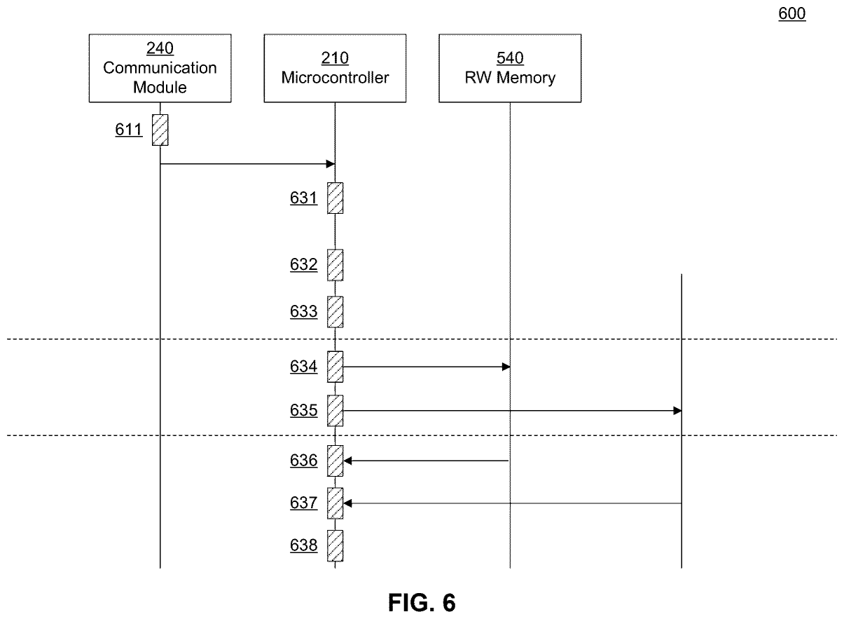

FIG. 6 is a diagram illustrating an example operational and data flow for over-the-air (“OTA”) programming of storage memory 5030 in analyte monitoring system 100 as well as use of the memory after the OTA programming in execution of processes by analyte sensor 110 according to the disclosed subject matter. In the example OTA programming 600 illustrated in FIG. 6, a request is sent from an external device (e.g., multi-purpose data receiving device 130) to initiate OTA programming (or re-programming). At 611, communication module 240 of analyte sensor 110 receives an OTA programming command. Communication module 240 sends the OTA programming command to the microcontroller 210 of analyte sensor 110.

At 631, after receiving the OTA programming command, microcontroller 210 validates the OTA programming command. Microcontroller 210 can determine, for example, whether the OTA programming command is signed with an appropriate digital signature token. Upon determining that the OTA programming command is valid, microcontroller 210 can set the sensor device into an OTA programming mode. At 632, microcontroller 210 can validate the OTA programming data. At 633, microcontroller 210 can reset analyte sensor 110 to re-initialize analyte sensor 110 in a programming state. Once analyte sensor 110 has transitioned into the OTA programming state, microcontroller 210 can begin to write data to the rewriteable memory (e.g., memory 5020) of the sensor device at 634 and write data to OTP memory 550 of the sensor device at 635 (e.g., storage memory 5030). The data written by the microcontroller 210 can be based on the validated OTA programming data. Microcontroller 210 can write data to cause one or more programming blocks or regions of OTP memory 550 to be marked invalid or inaccessible. The data written to the free or unused portion of OTP memory 550 can be used to replace invalidated or inaccessible programming blocks of OTP memory 550. After microcontroller 210 writes the data to the respective memories at 634 and 635, microcontroller 210 can perform one or more software integrity checks to ensure that errors were not introduced into the programming blocks during the writing process. Once microcontroller 210 is able to determine that the data has been written without errors, microcontroller 210 can resume standard operations of the sensor device.

In execution mode, at 636, microcontroller 210 can retrieve a programming manifest or profile from the rewriteable memory. The programming manifest or profile can include a listing of the valid software programming blocks and can include a guide to program execution for analyte sensor 110. By following the programming manifest or profile, microcontroller 210 can determine which memory blocks of OTP memory 550 are appropriate to execute and avoid execution of out-of-date or invalidated programming blocks or reference to out-of-date data. At 637, microcontroller 210 can selectively retrieve memory blocks from OTP memory 550. At 638, microcontroller 210 can use the retrieved memory blocks, by executing programming code stored or using variable stored in the memory.

### Exemplary Reader Device

FIG. 2A is a block diagram depicting an example embodiment of a reader device configured as a smartphone. Here, data receiving device 120 can include a display 122, input component 121, and processing core 206 including communications processor 222 coupled with memory 223 and applications processor 224 coupled with memory 225. Also included can be separate memory 230, RF transceiver 228 with antenna 229, and power supply 226 with power management module 238. Further included can be a multi-functional transceiver 232 which can communicate over Wi-Fi, NFC, Bluetooth, BTLE, and GPS with antenna 234. As understood by one of skill in the art, these components are electrically and communicatively coupled in a manner to make a functional device.

### Exemplary Data Receiving Device Architecture

For purpose of illustration and not limitation, reference is made to the exemplary embodiment of data receiving device 120 for use with the disclosed subject matter as shown in FIG. 2B. Data receiving device 120 and the related multi-purpose data receiving device 130 include components germane to the discussion of analyte sensor 110 and its operations and additional components can be included. In particular embodiments, data receiving device 120 and multi-purpose data receiving device 130 can be or include components provided by a third party and are not necessarily restricted to include devices made by the same manufacturer as analyte sensor 110.

As illustrated in FIG. 2B, data receiving device 120 includes ASIC 4000 having microcontroller 4010, memory 4020, and storage 4030 and communicatively coupled with a communication module 4040. Power for the components of data receiving device 120 can be delivered by power module 4050, which as embodied herein can include a rechargeable battery. Data receiving device 120 can further include display 4070 for facilitating review of analyte data received from analyte sensor 110 or other device (e.g., user device 140 or remote application server 150). Data receiving device 120 can include separate user interface components (e.g., physical keys, light sensors, microphones, etc.).

Communication module 4040 can include BLE module 4041 and NFC module 4042. Data receiving device 120 can be configured to wirelessly couple with analyte sensor 110 and transmit commands to and receive data from analyte sensor 110. As embodied herein, data receiving device 120 can be configured to operate, with respect to analyte sensor 110 as described herein, as an NFC scanner and a BLE end point via specific modules (e.g., BLE module 4042 or NFC module 4043) of communication module 4040. For example, data receiving device 120 can issue commands (e.g., activation commands for a data broadcast mode of the sensor; pairing commands to identify data receiving device 120) to analyte sensor 110 using a first module of the communication module 4040 and receive data from and transmit data to analyte sensor 110 using a second module of the communication module 4040. Data receiving device 120 can be configured for communication with user device 140 via a Universal Serial Bus (“USB”) module 4045 of the communication module 4040.

As another example, communication module 4040 can include, for example, cellular radio module 4044. The cellular radio module 4044 can include one or more radio transceivers for communicating using broadband cellular networks, including, but not limited to third generation (“3G”), fourth generation (“4G”), and fifth generation (“5G”) networks. Additionally, communication module 4040 of data receiving device 120 can include Wi-Fi radio module 4043 for communication using a wireless local area network according to one or more of the IEEE 802.11 standards (e.g., 802.11a, 802.11b, 802.11g, 802.11n (aka Wi-Fi 4), 802.11ac (aka Wi-Fi 5), 802.11ax (aka Wi-Fi 6)). Using cellular radio module 4044 or Wi-Fi radio module 4043, data receiving device 120 can communicate with remote application server 150 to receive analyte data or provide updates or input received from a user (e.g., through one or more user interfaces). Although not illustrated, communication module 5040 of analyte sensor 110 can similarly include a cellular radio module or Wi-Fi radio module.

As embodied herein, on-board storage 4030 of data receiving device 120 can store analyte data received from analyte sensor 110. Further, data receiving device 120, multi-purpose data receiving device 130, or user device 140 can be configured to communicate with remote application server 150 via a wide area network. As embodied herein, analyte sensor 110 can provide data to data receiving device 120 or multi-purpose data receiving device 130. Data receiving device 120 can transmit the data to user device 140. User device 140 (or multi-purpose data receiving device 130) can in turn transmit that data to remote application server 150 for processing and analysis.

As embodied herein, data receiving device 120 can further include sensing hardware 4060 similar to, or expanded from, sensing hardware 5060 of analyte sensor 110. In particular embodiments, data receiving device 120 can be configured to operate in coordination with analyte sensor 110 and based on analyte data received from the analyte sensor 110. As an example, where analyte sensor 110 is a glucose sensor, data receiving device 120 can be or include an insulin pump or insulin injection pen. In coordination, multi-purpose data receiving device 130 can adjust an insulin dosage for a user based on glucose values received from the analyte sensor.

### Exemplary Sensor Control Devices

FIGS. 2C and 2D are block diagrams depicting example embodiments of sensor control device 102 having in vivo analyte sensor 104 and sensor electronics 169 (including analyte monitoring circuitry) that can have the majority of the processing capability for rendering end-result data suitable for display to the user. In FIG. 2C, a single semiconductor chip (“ASIC”) 161 is depicted that can be a custom application specific integrated circuit. Shown within ASIC 161 are certain high-level functional units, including an analog front end (“AFE”) 162, power management circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

Memory 163 is also included within ASIC 161 and can be shared by the various functional units present within ASIC 161, or can be distributed amongst two or more of them. Memory 163 can also be a separate chip. Memory 163 can be volatile and/or non-volatile memory. In this embodiment, ASIC 161 is coupled with power source 170, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to data receiving device 120 (not shown), for example, where minimal further processing is needed by the resident software application to display the data.

FIG. 2D is similar to FIG. 2C but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 includes memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

For purpose of illustration and not limitation, reference is made to the exemplary embodiment of analyte sensor 110 for use with the disclosed subject matter as shown in FIG. 2E. FIG. 2E illustrates a block diagram of an example analyte sensor 110 according to exemplary embodiments compatible with the security architecture and communication schemes described herein.

As embodied herein, analyte sensor 110 can include ASIC 5000 communicatively coupled with a communication module 5040. ASIC 5000 can include a microcontroller core 5010, on-board memory 5020, and storage memory 5030. Storage memory 5030 can store data used in an authentication and encryption security architecture. Storage memory 5030 can store programming instructions for analyte sensor 110. As embodied herein, certain communication chipsets can be embedded in the ASIC 5000 (e.g., an NFC transceiver 5025). ASIC 5000 can receive power from a power module 5050, such as an on-board battery or from an NFC pulse. Storage memory 5030 of the ASIC 5000 can be programmed to include information such as an identifier for analyte sensor 110 for identification and tracking purposes. Storage memory 5030 can also be programmed with configuration or calibration parameters for use by analyte sensor 110 and its various components. Storage memory 5030 can include rewritable or one-time programming (“OTP”) memory. The storage memory 5030 can be updated using techniques described herein to extend the usefulness of analyte sensor 110.

As embodied herein, communication module 5040 of sensor 110 can be or include one or more modules to support analyte sensor 110 communicating with other devices of the analyte monitoring system 100. As an example only and not by way of limitation, example communication modules 5040 can include BLE module 5041 As used throughout this disclosure, BLE refers to a short-range communication protocol optimized to make pairing of Bluetooth devices simple for end users. Communication module 5040 can transmit and receive data and commands via interaction with similarly-capable communication modules of data receiving device 120 or user device 140. Communication module 5040 can include additional or alternative chipsets for use with similar short-range communication schemes, such as a personal area network according to IEEE 802.15 protocols, IEEE 802.11 protocols, infrared communications according to the Infrared Data Association standards (IrDA), etc.

To perform its functionalities, sensor 110 can further include suitable sensing hardware 5060 appropriate to its function. As embodied herein, sensing hardware 5060 can include an analyte sensor transcutaneously or subcutaneously positioned in contact with a bodily fluid of a subject. The analyte sensor can generate sensor data containing values corresponding to levels of one or more analytes within the bodily fluid.

### Exemplary Assembly Processes for Sensor Control Devices

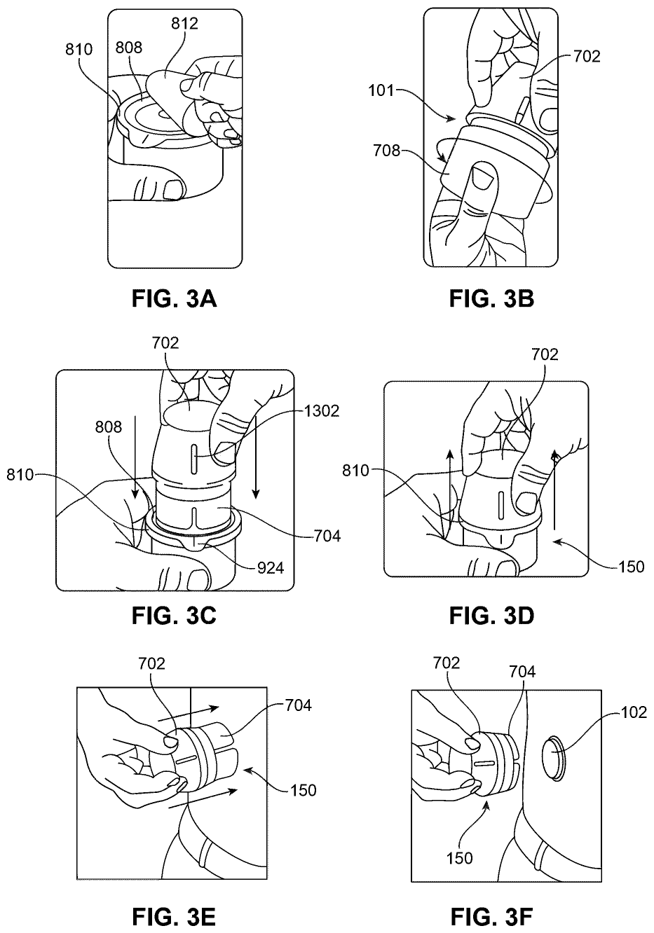

The components of sensor control device 102 can be acquired by a user in multiple packages requiring final assembly by the user before delivery to an appropriate user location. FIGS. 3A-3D depict an example embodiment of an assembly process for sensor control device 102 by a user, including preparation of separate components before coupling the components in order to ready the sensor for delivery. FIGS. 3E-3F depict an example embodiment of delivery of sensor control device 102 to an appropriate user location by selecting the appropriate delivery location and applying device 102 to the location.

FIG. 3A is a proximal perspective view depicting an example embodiment of user preparing tray 810, configured here as a tray (although other packages/containers can be used), for an assembly process. The user can accomplish this preparation by removing lid 812 from tray 810 to expose platform 808, for instance by peeling a non-adhered portion of lid 812 away from tray 810 such that adhered portions of lid 812 are removed. Removal of lid 812 can be appropriate in various embodiments so long as platform 808 is adequately exposed within tray 810. Lid 812 can then be placed aside.

FIG. 3B is a side view depicting an example embodiment of a user preparing a sensor applicator 101 for assembly. Sensor applicator 101 can be provided in a sterile package sealed by a cap 708. Preparation of sensor applicator 101 can include uncoupling housing 702 from cap 708 to expose sheath 704 (FIG. 3C). This can be accomplished by unscrewing (or otherwise uncoupling) cap 708 from housing 702. Cap 708 can then be placed aside.

FIG. 3C is a proximal perspective view depicting an example embodiment of a user inserting sensor applicator 101 into tray 810 during an assembly. Initially, the user can insert sheath 704 into platform 808 inside tray 810 after aligning a housing orienting feature (or slot or recess) and tray orienting feature 924 (an abutment or detent). Inserting sheath 704 into platform 808 temporarily unlocks sheath 704 relative to housing 702 and also temporarily unlocks platform 808 relative to tray 810. At this stage, removal of sensor applicator 101 from tray 810 will result in the same state prior to initial insertion of sensor applicator 101 into tray 810 (i.e., the process can be reversed or aborted at this point and then repeated without consequence).

Sheath 704 can maintain position within platform 808 with respect to housing 702 while housing 702 is distally advanced, coupling with platform 808 to distally advance platform 808 with respect to tray 810. This step unlocks and collapses platform 808 within tray 810. Sheath 704 can contact and disengage locking features (not shown) within tray 810 that unlock sheath 704 with respect to housing 702 and prevent sheath 704 from moving (relatively) while housing 702 continues to distally advance platform 808. At the end of advancement of housing 702 and platform 808, sheath 704 is permanently unlocked relative to housing 702. A sharp and sensor (not shown) within tray 810 can be coupled with an electronics housing (not shown) within housing 702 at the end of the distal advancement of housing 702. Operation and interaction of the sensor applicator 101 and tray 810 are further described below.

FIG. 3D is a proximal perspective view depicting an example embodiment of a user removing a sensor applicator 101 from tray 810 during an assembly. A user can remove sensor applicator 101 from tray 810 by proximally advancing housing 702 with respect to tray 810 or other motions having the same end effect of uncoupling sensor applicator 101 and tray 810. The sensor applicator 101 is removed with sensor control device 102 (not shown) fully assembled (sharp, sensor, electronics) therein and positioned for delivery.

FIG. 3E is a proximal perspective view depicting an example embodiment of a patient applying sensor control device 102 using sensor applicator 101 to a target area of skin, for instance, on an abdomen or any other appropriate location (e.g., on a limb). Advancing housing 702 distally collapses sheath 704 within housing 702 and applies the sensor to the target location such that an adhesive layer on the bottom side of sensor control device 102 adheres to the skin. The sharp is automatically retracted when housing 702 is fully advanced, while the sensor (not shown) is left in position to measure analyte levels.

FIG. 3F is a proximal perspective view depicting an example embodiment of a patient with sensor control device 102 in an example applied position. The user can then remove sensor applicator 101 from the application site.

Analyte monitoring system 100, described with respect to FIGS. 3A-3F and elsewhere herein, can provide a reduced or eliminated chance of accidental breakage, permanent deformation, or incorrect assembly of applicator components compared to prior art systems. Since housing 702 directly engages platform 808 while sheath 704 unlocks, rather than indirect engagement via sheath 704, relative angularity between sheath 704 and housing 702 will not result in breakage or permanent deformation of the arms or other components. The potential for relatively high forces (such as in conventional devices) during assembly will be reduced, which in turn reduces the chance of unsuccessful user assembly.

### Exemplary Sensor Applicators

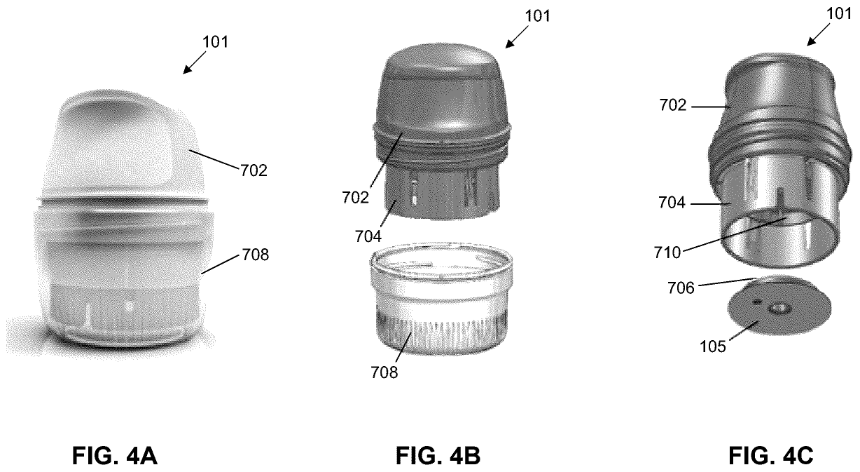

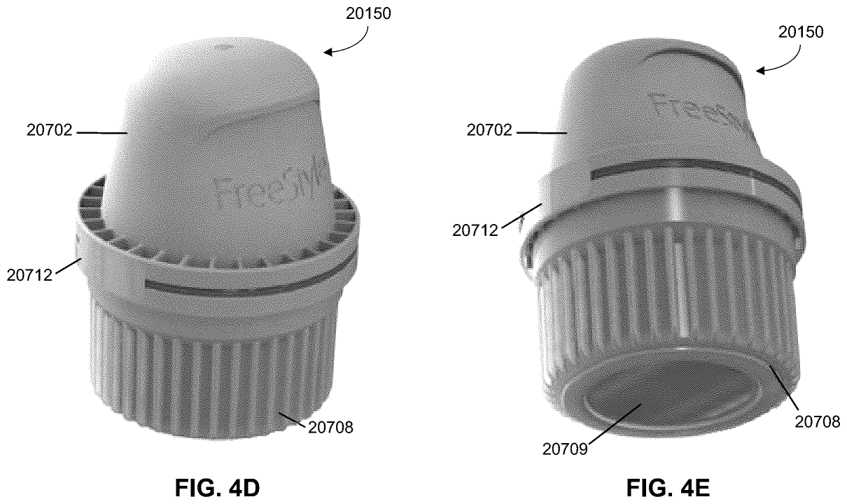

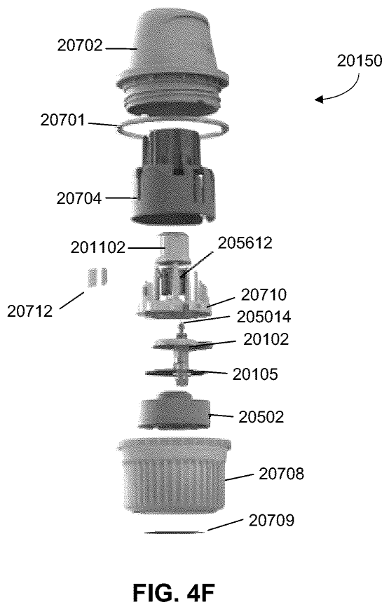

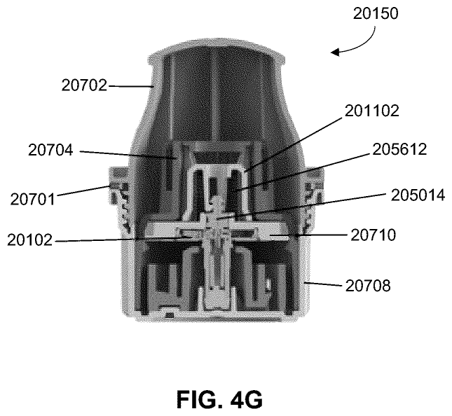

FIG. 4A is a side view depicting an example embodiment of a sensor applicator 101 coupled with screw cap 708. This is an example of how sensor applicator 101 is shipped to and received by a user, prior to assembly by the user with a sensor. FIG. 4B is a side perspective view depicting sensor applicator 101 and cap 708 after being decoupled. FIG. 4C is a perspective view depicting an example embodiment of a distal end of sensor applicator 101 with electronics housing 706 and adhesive patch 105 removed from the position they would have retained within sensor carrier 710 of sheath 704, when cap 708 is in place.

Referring to FIG. 4D-G for purpose of illustration and not limitation, sensor applicator 20150 can be provided to a user as a single integrated assembly. FIGS. 4D and 4E provide perspective top and bottom views, respectively, of the sensor applicator 20150, FIG. 4F provides an exploded view of sensor applicator 20150 and FIG. 4G provides a side cut-away view. The perspective views illustrate how sensor applicator 20150 is shipped to and received by a user. The exploded and cut-away views illustrate the components of sensor applicator 20150. The sensor applicator 20150 can include a housing 20702, gasket 20701, sheath 20704, sharp carrier 201102, spring 205612, sensor carrier 20710 (also referred to as a “puck carrier”), sharp hub 205014, sensor control device (also referred to as a “puck”) 20102, adhesive patch 20105, desiccant 20502, cap 20708, serial label 20709, and tamper evidence feature 20712. As received by a user, only housing 20702, cap 20708, tamper evidence feature 20712, and label 20709 are visible. The tamper evidence feature 20712 can be, for example, a sticker coupled to each of housing 20702 and cap 20708, and tamper evidence feature 20712 can be damaged, for example, irreparably, by uncoupling housing 20702 and cap 20708, thereby indicating to a user that housing 20702 and cap 20708 have been previously uncoupled. These features are described in greater detail below.

### Exemplary Sensor States and Activation

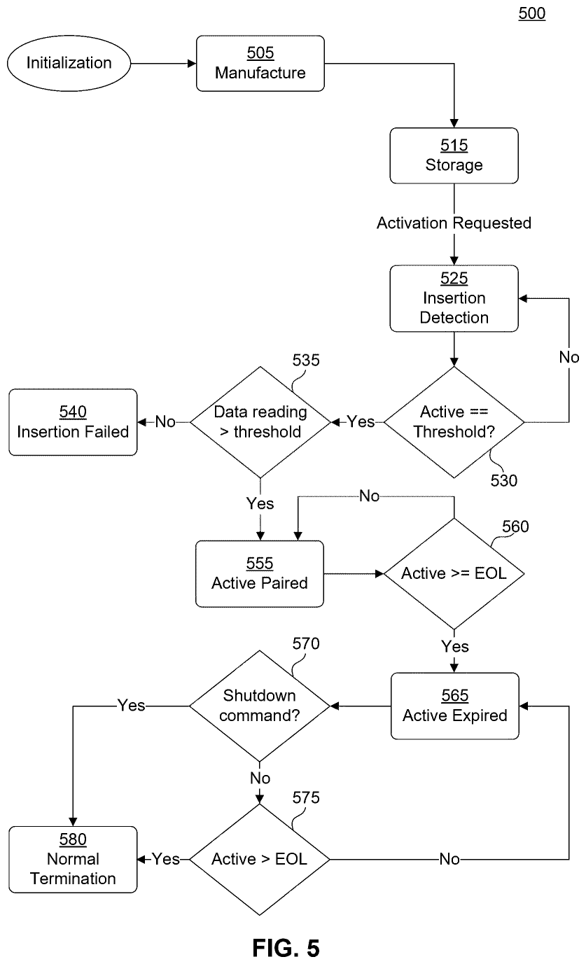

For purpose of illustration and not limitation, reference is made to the exemplary embodiment of a high-level depiction of a state machine representation 500 of the actions that can be taken by analyte sensor 110 as shown in FIG. 5. After initialization, the sensor enters state 505, which relates to the manufacture of analyte sensor 110. In the manufacture state 505, analyte sensor 110 can be configured for operation, for example, the storage memory 5030 can be written. At various times while in state 505, analyte sensor 110 checks for a received command to go to storage state 515. Upon entry to storage state 515, the sensor performs a software integrity check. While in storage state 515, the sensor can also receive an activation request command before advancing to insertion detection state 525.

Upon entry to state 525, analyte sensor 110 can store information relating to devices authenticated to communicate with the sensor as set during activation or initialize algorithms related to conducting and interpreting measurements from sensing hardware 5060. Analyte sensor 110 can also initialize a lifecycle timer, responsible for maintaining an active count of the time of operation of analyte sensor 110 and begin communication with authenticated devices to transmit recorded data. While in insertion detection state 525, the sensor can enter state 530, where analyte sensor 110 checks whether the time of operation is equal to a predetermined threshold. This time of operation threshold can correspond to a timeout function for determining whether an insertion has been successful. If the time of operation has reached the threshold, analyte sensor 110 advances to state 535, in which analyte sensor 110 checks whether the average data reading is greater than a threshold amount corresponding to an expected data reading volume for triggering detection of a successful insertion. If the data reading volume is lower than the threshold while in state 535, the sensor advances to state 540, corresponding to a failed insertion. If the data reading volume satisfies the threshold, the sensor advances to active paired state 555.

Active paired state 555 of analyte sensor 110 reflects the state while analyte sensor 110 is operating as normal by recording measurements, processing the measurements, and reporting them as appropriate. While in active paired state 555, analyte sensor 110 sends measurement results or attempts to establish a connection with data receiving device 120. Analyte sensor 110 also increments the time of operation. Once analyte sensor 110 reaches a predetermined threshold time of operation (e.g., once the time of operation reaches a predetermined threshold), analyte sensor 110 transitions to active expired state 565. Active expired state 565 of analyte sensor 110 reflects the state while analyte sensor 110 has operated for its maximum predetermined amount of time.

While in active expired state 565, analyte sensor 110 can generally perform operations relating to winding down operation and ensuring that the collected measurements have been securely transmitted to receiving devices as needed. For example, while in active expired state 565, analyte sensor 110 can transmit collected data and, if no connection is available, can increase efforts to discover authenticated devices nearby and establish and connection therewith. While in active expired state 565, analyte sensor 110 can receive a shutdown command at state 570. If no shutdown command is received, analyte sensor 110 can also, at state 575, check if the time of operation has exceeded a final operation threshold. The final operation threshold can be based on the battery life of analyte sensor 110. The normal termination state 580 corresponds to the final operations of analyte sensor 110 and ultimately shutting down analyte sensor 110.

Before a sensor is activated, ASIC 5000 resides in a low power storage mode state. The activation process can begin, for example, when an incoming RF field (e.g., NFC field) drives the voltage of the power supply to the ASIC 5000 above a reset threshold, which causes analyte sensor 110 to enter a wake-up state. While in the wake-up state, ASIC 5000 enters an activation sequence state. ASIC 5000 then wakes communication module 5040. Communication module 5040 is initialized, triggering a power on self-test. The power on self-test can include ASIC 5000 communicating with communication module 5040 using a prescribed sequence of reading and writing data to verify the memory and one-time programmable memory are not corrupted.

When ASIC 5000 enters the measurement mode for the first time, an insertion detection sequence is performed to verify that analyte sensor 110 has been properly installed onto the patient's body before a proper measurement can take place. First, analyte sensor 110 interprets a command to activate the measurement configuration process, causing the ASIC 5000 to enter measurement command mode. Analyte sensor 110 then temporarily enters the measurement lifecycle state to run a number of consecutive measurements to test whether the insertion has been successful. Communication module 5040 or ASIC 5000 evaluates the measurement results to determine insertion success. When insertion is deemed successful, analyte sensor 110 enters a measurement state, in which analyte sensor 110 begins taking regular measurements using sensing hardware 5060. If analyte sensor 110 determines that the insertion was not successful, analyte sensor 110 is triggered into an insertion failure mode, in which ASIC 5000 is commanded back to storage mode while communication module 5040 disables itself.

### Exemplary Security and Other Architecture Features

As embodied herein a first layer of security for communications between analyte sensor 110 and other devices can be established based on security protocols specified by and integrated in the communication protocols used for the communication. Another layer of security can be based on communication protocols that necessitate close proximity of communicating devices. Furthermore certain packets and/or certain data included within packets can be encrypted while other packets and/or data within packets is otherwise encrypted or not encrypted. Additionally or alternatively, application layer encryption can be used with one or more block ciphers or stream ciphers to establish mutual authentication and communication encryption with other devices in the analyte monitoring system 100.

ASIC 5000 of analyte sensor 110 in FIG. 2E can be configured to dynamically generate authentication and encryption keys using data retained within storage memory 5030. Storage memory 5030 can also be pre-programmed with a set of valid authentication and encryption keys to use with particular classes of devices. ASIC 5000 can be further configured to perform authentication procedures with other devices using received data and apply the generated key to sensitive data prior to transmitting the sensitive data. The generated key can be unique to analyte sensor 110, unique to a pair of devices, unique to a communication session between analyte sensor 110 and other device, unique to a message sent during a communication session, or unique to a block of data contained within a message.

Both analyte sensor 110 and data receiving device 120 can ensure the authorization of the other party in a communication session to, for example, issue a command or receive data. In particular embodiments, identity authentication can be performed through two features. First, the party asserting its identity provides a validated certificate signed by the manufacturer of the device or the operator of analyte monitoring system 100. Second, authentication can be enforced through the use of public keys and private keys, and shared secrets derived therefrom, established by the devices of analyte monitoring system 100 or established by the operator of the analyte monitoring system 100. To confirm the identity of the other party, the party can provide proof that the party has control of its private key.

The manufacturer of analyte sensor 110, data receiving device 120, or provider of the application for multi-purpose data receiving device 130 can provide information and programming necessary for the devices to securely communicate through secured programming and updates. For example, the manufacturer can provide information that can be used to generate encryption keys for each device, including secured root keys for analyte sensor 110 and optionally for data receiving device 120 that can be used in combination with device-specific information and operational data (e.g., entropy-based random values) to generate encryption values unique to the device, session, or data transmission as need.

Analyte data associated with a user is sensitive data at least in part because this information can be used for a variety of purposes, including for health monitoring and medication dosing decisions. In addition to user data, analyte monitoring system 100 can enforce security hardening against efforts by outside parties to reverse-engineering. Communication connections can be encrypted using a device-unique or session-unique encryption key. Encrypted communications or unencrypted communications between any two devices can be verified with transmission integrity checks built into the communications. Analyte sensor 110 operations can be protected from tampering by restricting access to read and write functions to the memory 5020 via a communication interface. The sensor can be configured to grant access only to known or “trusted” devices, provided in a “whitelist” or only to devices that can provide a predetermined code associated with the manufacturer or an otherwise authenticated user. A whitelist can represent an exclusive range, meaning that no connection identifiers besides those included in the whitelist will be used, or a preferred range, in which the whitelist is searched first, but other devices can still be used. Analyte sensor 110 can further deny and shut down connection requests if the requestor cannot complete a login procedure over a communication interface within a predetermined period of time (e.g., within four seconds). These characteristics safeguard against specific denial of service attacks, and in particular against denial of service attacks on a BLE interface.

As embodied herein, analyte monitoring system 100 can employ periodic key rotation to further reduce the likelihood of key compromise and exploitation. A key rotation strategy employed by analyte monitoring system 100 can be designed to support backward compatibility of field-deployed or distributed devices. As an example, analyte monitoring system 100 can employ keys for downstream devices (e.g., devices that are in the field or cannot be feasibly provided updates) that are designed to be compatible with multiple generations of keys used by upstream devices.

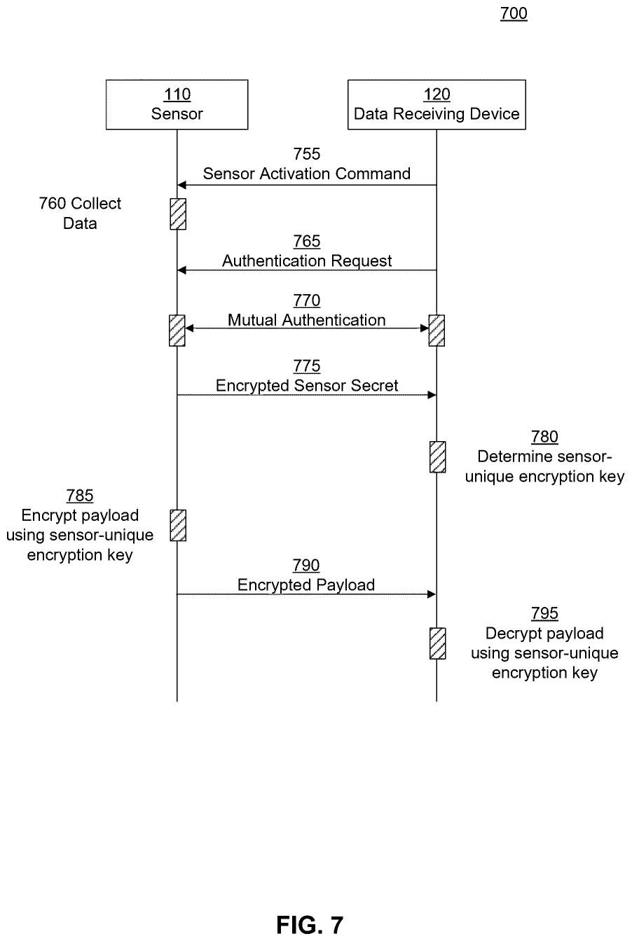

For purpose of illustration and not limitation, reference is made to the exemplary embodiment of a message sequence diagram for use with the disclosed subject matter as shown in FIG. 7 and demonstrating an example exchange of data between a pair of devices, particularly analyte sensor 110 and data receiving device 120. Data receiving device 120 can, as embodied herein, be data receiving device 120 or multi-purpose data receiving device 130. At step 755, data receiving device 120 can transmit a sensor activation command to analyte sensor 110, for example, via a short-range communication protocol. Analyte sensor 110 can, prior to step 755 be in a primarily dormant state, preserving its battery until full activation is needed. After activation during step 760, analyte sensor 110 can collect data or perform other operations as appropriate to the sensing hardware 5060 of analyte sensor 110. At step 765, data receiving device 120 can an initiate authentication request command. In response to the authentication request command, both analyte sensor 110 and data receiving device 120 can engage in a mutual authentication process in step 770. Step 770 can involve the transfer of data, including challenge parameters that allow analyte sensor 110 and data receiving device 120 to ensure that the other device is sufficiently capable of adhering to an agreed-upon security framework described herein. Mutual authentication can be based on mechanisms for authentication of two or more entities to each other with or without on-line trusted third parties to verify establishment of a secret key via challenge-response. Mutual authentication can be performed using two-, three-, four-, or five-pass authentication, or similar versions thereof.

Following a successful step 770, at step 775 analyte sensor 110 can provide data receiving device 120 with a sensor secret. The sensor secret can contain sensor-unique values and be derived from random values generated during manufacture. The sensor secret can be encrypted prior to or during transmission to prevent third-parties from accessing the secret. The sensor secret can be encrypted via one or more of the keys generated by or in response to the mutual authentication process. At step 780, data receiving device 120 can derive a sensor-unique encryption key from the sensor secret. The sensor-unique encryption key can further be session-unique. As such, the sensor-unique encryption key can be determined by each device without being transmitted between analyte sensor 110 or data receiving device 120. At step 785, analyte sensor 110 can encrypt data to be included in payload. At step 790, analyte sensor 110 can transmit the encrypted payload 740 to data receiving device 120 using the communication link established between the appropriate communication models of analyte sensor 110 and data receiving device 120. At step 795, data receiving device 120 can decrypt the payload using the sensor-unique encryption key derived during step 780. Following step 795, analyte sensor 110 can deliver additional (including newly collected) data and data receiving device 120 can process the received data appropriately.

As discussed herein, analyte sensor 110 can be a device with restricted processing power, battery supply, and storage. The encryption techniques used by analyte sensor 110 (e.g., the cipher algorithm or the choice of implementation of the algorithm) can be selected based at least in part on these restrictions. Data receiving device 120 can be a more powerful device with fewer restrictions of this nature. Therefore, data receiving device 120 can employ more sophisticated, computationally intense encryption techniques, such as cipher algorithms and implementations.

### Methods Allowing for More Frequent Titration

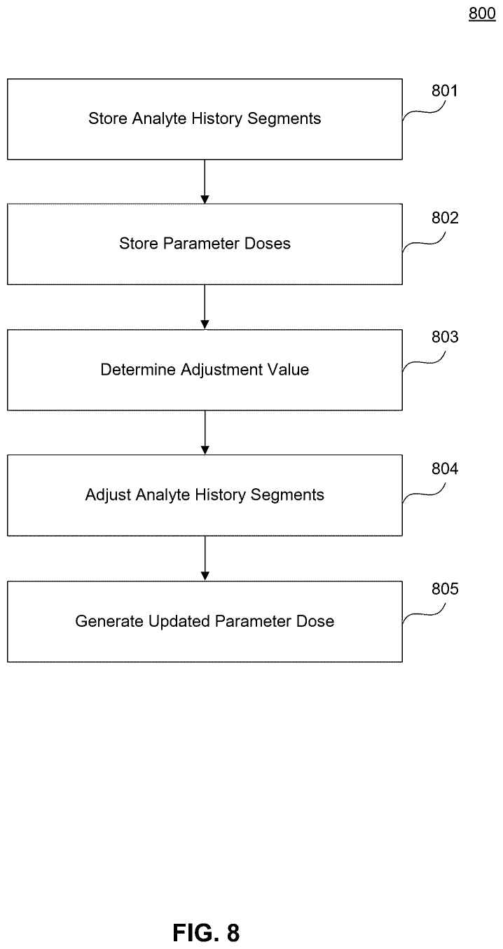

FIG. 8 is a flowchart illustrating a method 800 to negate the effect of past parameter doses on affected analyte history segments by adjusting the affected analyte history segment to compensate for the past parameter dose, according to some embodiments. Method 800 can be performed by processing logic that can comprise hardware (e.g., circuitry, dedicated logic, programmable logic, microcode, etc.), software (e.g., instructions executing on a processing device), or a combination thereof. Method 800 can be stored and/or executed on the phone, a medication delivery device (i.e., an insulin pen or cap), in the cloud, or any combination thereof. Method 800 can be performed using various titration algorithms, such as a generic titration algorithm based on the guidelines of the American Diabetes Association, a user specific personalized titration algorithm based on user biometrics, a titration algorithm generated by an artificial intelligence or machine learning process, or any combination thereof. In some embodiments, the user or system selects the preferred titration algorithm to use. It is to be appreciated that not all steps may be needed to perform the disclosure provided herein. Further, some of the steps may be performed simultaneously, or in a different order than shown in FIG. 8, as will be understood by a person of ordinary skill in the art(s).

In 801, the value of an analyte is stored across an analyte history segment. In some embodiments, this may be accomplished with a continuous analyte monitoring system. Examples of segments of analyte history include segments most relevant to fasting and/or night time, segments most relevant to pre-meal analyte levels, segments most relevant to post-meal analyte levels, segments most relevant to night time hypo-analyte risk, segments most relevant to post-meal hypo-analyte risk, segments most relevant to night time hyper-analyte risk, segments most relevant to post-meal hyper-analyte risk, segments most relevant to the determination of pre-meal parameter correction factor, and segments most relevant to the determination of post-meal parameter correction factor. The identified segments may be constructed from a single contiguous analyte history or multiple disparate analyte histories.

In certain embodiments, the value of an analyte is stored for at least two analyte history segments. In certain embodiments, a first analyte history segment represents analyte values for a first time period; a second analyte history segment represents analyte values for a second time period, and so on. In certain embodiments, the value of an analyte is stored for a multitude of analyte history segments. In certain embodiments, the analyte history segments are not reset when a parameter titration occurs.

Resetting an analyte history segment means removing all analyte values that otherwise should be previously included or analyzed, and restarting the process of filling the analyte history segment with data whose timestamp is later than the reset time. Removed analyte values are not included in the rolling window of doses, for example, the rolling window shown in FIG. 12A.

In certain embodiments, the analyte value stored is a glucose value. In certain embodiments, the glucose value stored is related to basal glucose level (e.g., the stable overnight concentration of glucose, the glucose history from midnight to 4 AM (or any other predetermined period of time) may be assigned as the segment most relevant to basal glucose). In certain embodiments, the glucose value stored is related to non-basal glucose levels.

In 802 the value of parameter doses associated with the analyte history segments of 801 is stored.

In some embodiments, the parameter is an insulin dose. In some embodiments, the parameter is a basal insulin dose (e.g., a slow-acting insulin dose or a pre-mixed insulin dose (a combination or slow-and rapid-acting insulin). In some embodiments, the basal insulin dose changes across the plurality of analyte history segments. In some embodiments, the parameter is the fixed dose element of non-basal insulin dose such as pre-meal breakfast fixed dose, pre-meal lunch fixed dose, pre-meal dinner fixed dose, pre-meal fixed small meal dose, pre-meal fixed medium meal dose, or pre-meal fixed large meal dose (e.g., rapid-acting insulin bolus/dose or a pre-mixed insulin bolus/dose). In some embodiments, the non-basal insulin parameter does not change across the plurality of analyte history segments.

Examples of parameters include quantifiable values such as the dose amount of the long-acting insulin dose, and the fixed dose element of a mealtime rapid-acting (bolus) insulin dose.

Specific treatment choices, such as the specific choice of the long-acting insulin (e.g., degludec, glargine, detemir, icodec), the specific choice of the rapid-acting insulin (e.g., lispro, aspart, fiasp), the specific choice of a different type of insulin (e.g., pre-mixed, NPH), and the specific choice of other medication classes (e.g., biguanides, SGLT-2i, GLP-1Ra, DPP-4i, amylin analogues) can also be considered as a parameter by enumerating them.

For example, the choice of using or not using a particular treatment can be enumerated into 0 or 1, respectively. Furthermore, the choice can be enumerated into more than 2 values, corresponding to available dose options. For example, the choice of none, 0.25 mg, 0.5 mg, 1 mg, 1.7 mg, or 2.4 mg semaglutide dose can be enumerated into parameter values of 0, 1, 2, 3, 4, & 5 in that same order.

In 803 the data stored during method steps 801 and 802 are used to determine an adjustment value.

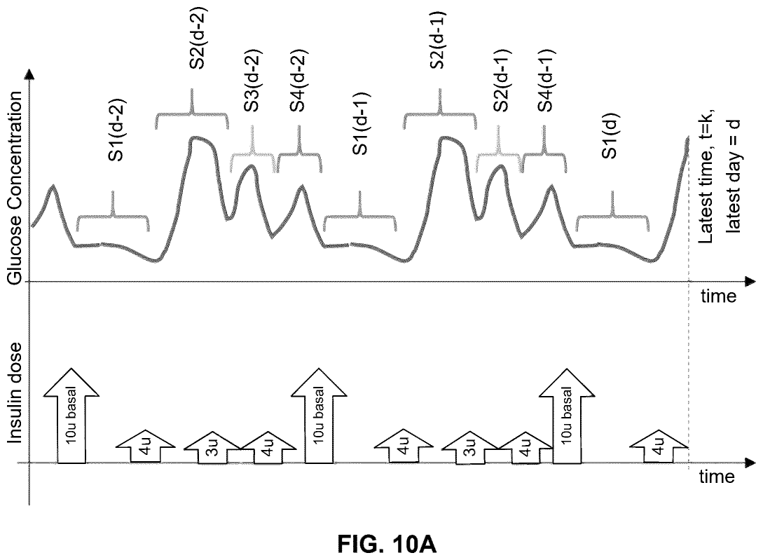

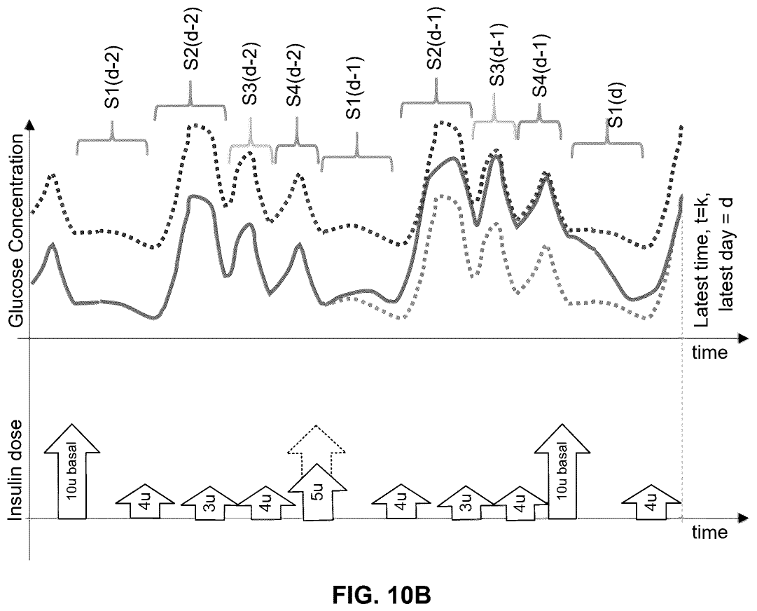

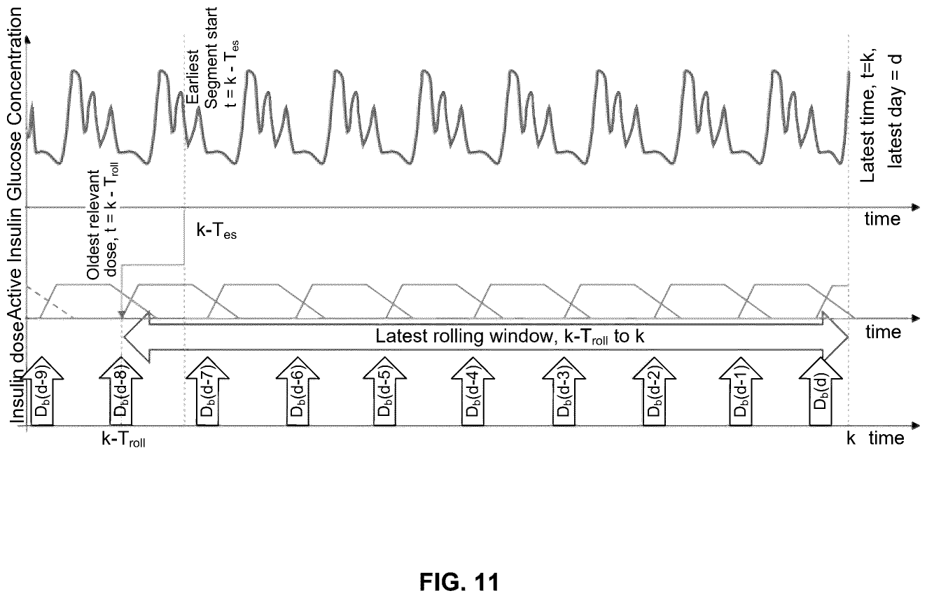

The value of an analyte stored across an analyte history segment under method step 801 and the value of a parameter doses associated with the analyte history segments of 801 stored under method step 802 are correlated with each other. An exemplary correlation of these two values where the parameter is insulin dose and the analyte is glucose is shown in FIGS. 10A and 10B. In FIG. 10A the insulin dose remains constant, whereas in FIG. 10B the insulin dose does not remain constant. FIG. 10B shows as dotted lines the hypothetical glucose concentrations caused by a constant insulin dose of either the 10 u (bottom dotted line) or 5 u (top dotted line) doses. In FIGS. 10A and 10B, S1 are segments for titrating basal dose, S2 are segments for titrating breakfast-related dose, S3 are segments for titrating lunch-related dose, and S4 are segments for titrating dinner-related dose. The first segment of a day corresponds to the segment for titrating basal (S1) dose. Note that a particular glucose value may belong to more than one segment. Segments can be defined temporally in multiple ways (i.e., a segment can be defined as being temporally related to the latest recorded day, d; a day at least one day before the latest recorded day, d−n (where n is the number of days from the latest day); a time in hours, t, before a most recent time point, wherein k is the most recent time point and k−t is one or more hours before the most recent time point.) Segments can be defined temporally in other ways (i.e., measured in minutes before the most recent time point, seconds before the most recent time point, weeks before the most recent time point, etc.).

Where the value of two parameter doses are equivalent, as shown in FIG. 11 where the parameter is insulin and the analyte is glucose, the adjustment value is zero,. FIG. 11 shows administration with a constant insulin dose, on the bottom graphic with time segments being temporally defined in terms of days before latest day. FIG. 11 shows the active insulin resulting from administration with a constant insulin dose on the middle segment. Active insulin associated with a particular insulin dose increases rapidly after administration, then remains constant for a time before decreasing to zero. Insulin associated with two subsequent insulin doses may be active in the body at the same time, as represented by the overlapping lines in the middle graphic of FIG. 11. In certain embodiments, insulin associated with two or more subsequent insulin doses may be active in the body at the same time. In certain embodiments, a parameter associated with two or more subsequent parameter doses may be active in the body at the same time. More complex or simpler temporal models of active insulin may be used. An example of a more complex temporal model of active insulin can be obtained by fitting a model with a pharmacokinetic/pharmacodynamic clinical data. Examples of simpler models maybe a simple rectangular shape in time or a triangular shape in time, both allowing active insulin to be present for a period of time.

FIG. 11 shows the body glucose concentration as a result of the administration with a constant insulin dose on the top graphic. FIG. 11 shows a “k-tₑₛ” which is the earliest analyte history segment of interest. FIG. 11 shows a “k-Tᵣₒₗₗ” which is the oldest dose that affects the analyte values recorded in k-tₑₛ, because the insulin from the k-Tᵣₒₗₗ dose is still active in the body of the user during a portion within k-tₑₛ. The dashed line on FIG. 11 indicates the effect of an insulin dose which no longer affects the body and is no longer relevant in the glucose value of the earliest glucose history segment of interest.

Where the value of two parameter doses are non-equivalent, as shown in FIG. 12A where the parameter is insulin and the analyte is glucose, the adjustment value is non-zero,. FIG. 12A shows administration with a non-constant insulin dose, on the bottom graphic with time segments being temporally defined in terms of days before latest day. FIG. 12A shows the active insulin resulting from administration with a non-constant insulin dose on the middle segment. Active insulin associated with a particular insulin dose increases rapidly after administration, then remains constant for a time before decreasing to zero. Insulin associated with two subsequent insulin doses may be active in the body at the same time, as represented by the overlapping lines in the middle graphic of FIG. 12A. In certain embodiments, insulin associated with two or more subsequent insulin doses may be active in the body at the same time. In certain embodiments, a parameter associated with two or more subsequent parameter doses may be active in the body at the same time. FIG. 12A shows the body glucose concentration as a result of the administration with a non-constant insulin dose on the top graphic. The dashed line on FIG. 12A indicates the effect of an insulin dose which no longer affects the body and is no longer relevant in the glucose value of the earliest glucose history segment of interest. The dotted line on FIG. 12A indicates the hypothetical active insulin if all insulin doses were equivalent to the most recent insulin dose.

FIG. 12A shows a “k−Tᵣₒₗₗ” which is the oldest dose that affects the analyte values recorded in k−tₑₛ. The “latest rolling window” covers a time period from k−Tᵣₒₗₗ to k. Glucose history within this rolling window may require adjustment when one or more titrations occur within the window. In certain embodiments, the rolling window needs to be adjusted whenever a new titration occurs. In certain embodiments, this adjustment may occur at one or more fixed times of day, occur dynamically upon a triggering event, or a combination of both. In some embodiments, when the requirements for another basal dose titration is met adjustment to the data contained in the latest rolling window is necessary to prevent erroneous titration decisions caused by glucose history in the latest rolling window being affected by dose amounts that may be different from the latest basal insulin dose (D_{b}(d)).

In some embodiments, as shown in FIG. 12A, several basal dose titration changes occur within the latest rolling window (i.e., down titrations at d-8 and d-4, and up titrations at d-5 and d-2). In FIG. 12A, there are 3 different dose amounts as denoted by the 3 dotted horizontal lines on the Insulin dose plot. This results in varying active insulin contributions, with the dotted traces in the active insulin plot corresponding to the middle dose for visual reference. The dotted trace in the glucose concentration plot shows what the glucose history looks like if the doses are kept at the hypothetical fixed value of this example, and the solid trace illustrates the actual glucose history as a result of the changing basal doses.

Next, the value of the most recent parameter dose is identified. Then the value of a parameter dose not associated with the most recent analyte history segment is subtracted from the value of the most recent parameter dose. This equation is exemplified in FIG. 12B.

$$Δ {D}_{b} ( d - i ) = {D}_{b} ( d ) - {D}_{b} ( d - i )$$

wherein D_{b}(d) is the value of the most recent parameter dose, D_{b}(d−i) is the value of the parameter dose “i” analyte history segments before the most recent segment, and ΔD_{b}(d−i) is the difference between the two. This calculation yields the difference between the most recent parameter dose and a historic parameter dose.

Next, the value of the difference in the parameter dose is used to generate an adjustment value, wherein the adjustment value is the analyte value associated with an administration of a hypothetical parameter dose equivalent to the value of the difference previously calculated.

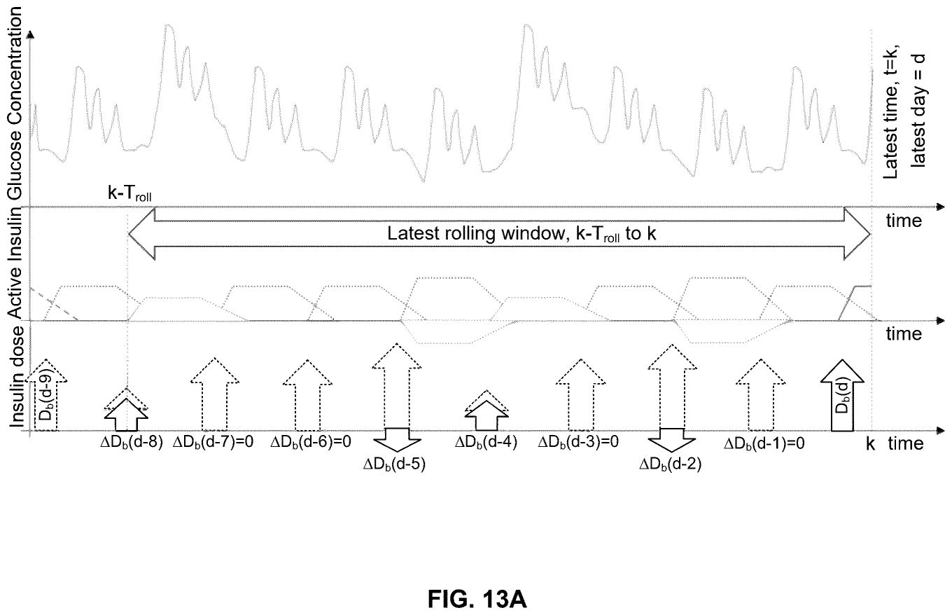

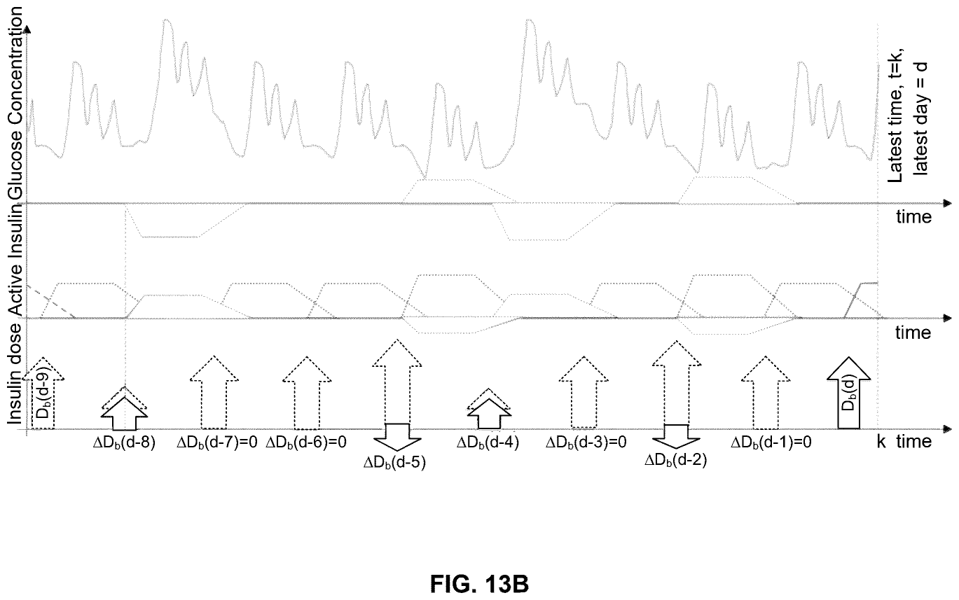

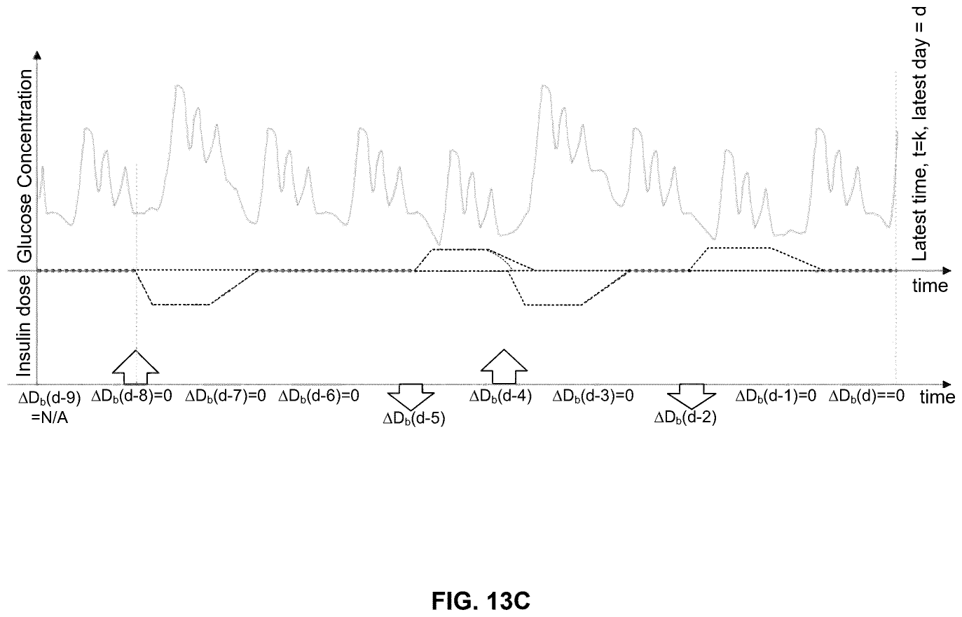

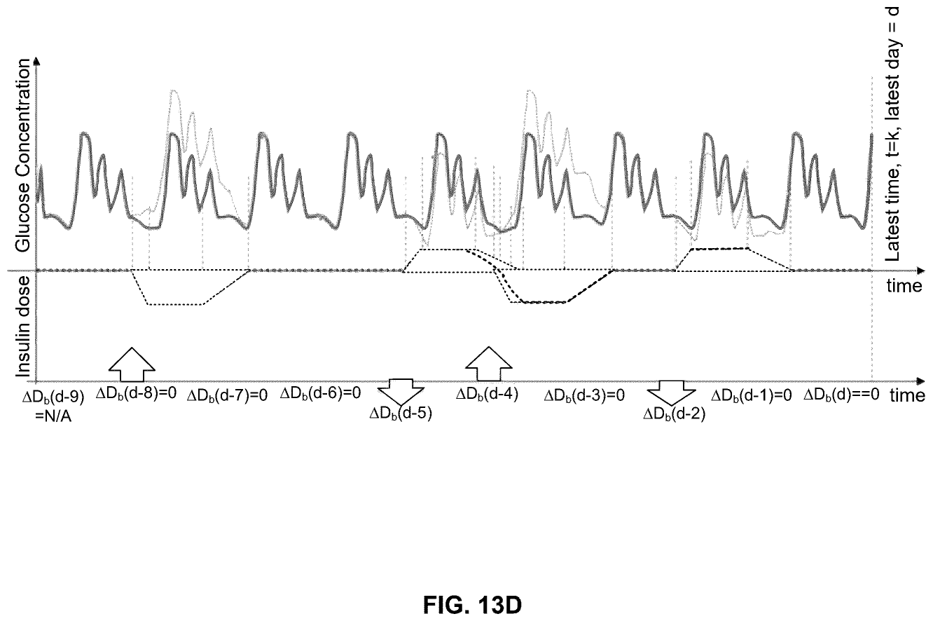

In some embodiments, the adjustment value can be determined according to the following method:
determining the hypothetical change in active parameter caused by a parameter dose of ΔD_{b}(d−i), as shown in FIG. 13A wherein the parameter is insulin;
determining the hypothetical change in analyte concentration caused by the hypothetical change in active parameter of step 1, as shown in FIG. 13B, wherein the analyte is glucose and the parameter is insulin;
summing the individual hypothetical analyte concentration changes together, as shown in FIG. 13C, wherein the analyte is glucose.

In some embodiments, the patient's previous response to the parameter is considered in calculating the adjustment value. In some embodiments, the patient's sensitivity to the parameter is considered in calculating the adjustment value. In some embodiments, one or more of the patient's carbohydrate-to-parameter ratio, the patient's pre-meal correction factor, the patient's post-meal correction factor, the parameter action time, the desired dosing time of day for the long-acting parameter dose, the patient's state of health, the patient's level of physical activity, and the patient's meal consumption pattern is considered in calculating the adjustment value.

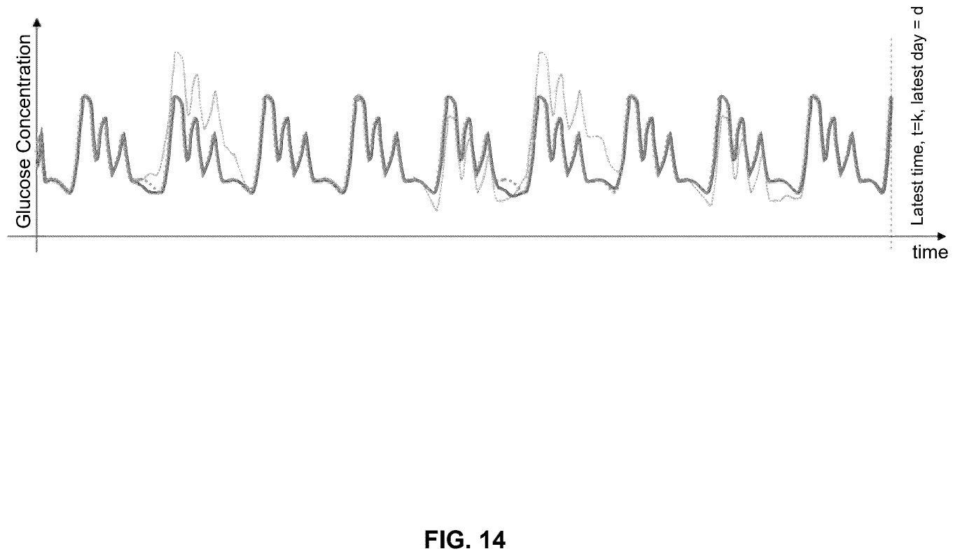

In 804, analyte values of the analyte history segments are adjusted to the values they would have been if the most recently administered parameter dose was administered during all the previous analyte history segments regardless of actual past parameter doses, based on the calculated adjustment value from method step 803. A graphical depiction of this adjustment being carried out is exemplified in FIG. 13D. This adjustment results in a continuous analyte history that is nearly identical to the hypothetical analyte history if all parameter doses were equivalent as shown by FIG. 14. In some embodiments, the analyte is glucose. In some embodiments, the analyte is basal glucose. In some embodiments, the parameter is an insulin dose. In some embodiments, the parameter is a basal insulin dose. In some embodiments, unknown factors such as changes in state of health, level of physical activity, and meal consumption pattern results in a mismatch between the adjusted glucose history and the hypothetical glucose history. In some embodiments, the mismatch between the adjusted glucose history and the hypothetical glucose history can be isolated in time areas where the effect of one dose overlaps with its adjacent dose In some embodiments, the mismatch between the adjusted glucose history and the hypothetical glucose history can affect the mean and/or dynamic range of the adjusted glucose history.

In 805, an updated parameter dose is generated based on the adjusted analyte history segments generated by method step 804. In some embodiments, the analyte is glucose. In some embodiments, the analyte is basal glucose. In some embodiments, the parameter is an insulin dose. In some embodiments, the parameter is a basal insulin dose.

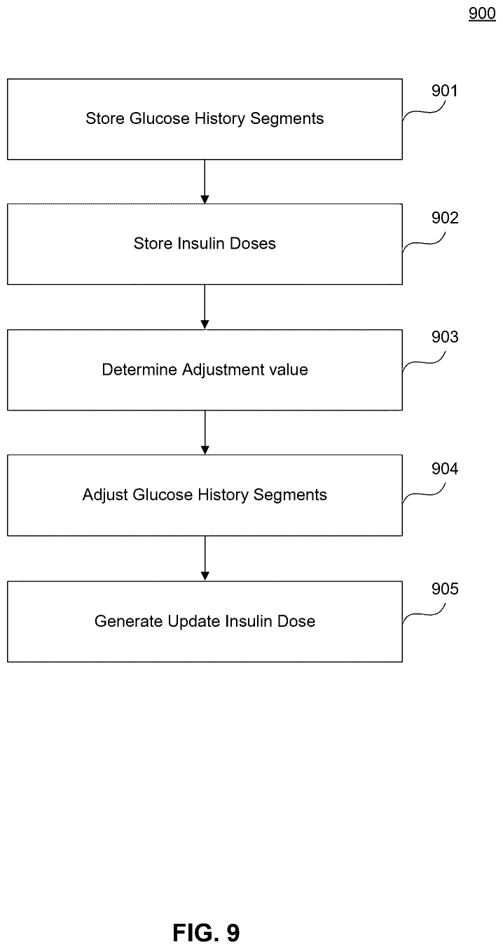

FIG. 9 is a flowchart illustrating a method 900 to negate the effect of past insulin doses on affected glucose history segments by adjusting the affected glucose history segment to compensate for the past insulin dose, according to some embodiments. Method 900 can be performed by processing logic that can comprise hardware (e.g., circuitry, dedicated logic, programmable logic, microcode, etc.), software (e.g., instructions executing on a processing device), or a combination thereof. Method 900 can be stored and/or executed on the phone, a medication delivery device (i.e., an insulin pen or cap), in the cloud, or any combination thereof. Method 900 can be performed using various titration algorithms, such as a generic titration algorithm based on the guidelines of the American Diabetes Association, a user specific personalized titration algorithm based on user biometrics, a titration algorithm generated by an artificial intelligence or machine learning process, or any combination thereof. In some embodiments, the user or system selects the preferred titration algorithm to use. It is to be appreciated that not all steps may be needed to perform the disclosure provided herein. Further, some of the steps may be performed simultaneously, or in a different order than shown in FIG. 9, as will be understood by a person of ordinary skill in the art(s).

In 901, the value of glucose is stored across a glucose history segment. In some embodiments, this may be accomplished with a continuous analyte monitoring system. Examples of segments of analyte history include segments most relevant to fasting and/or night time, segments most relevant to pre-meal glucose, segments most relevant to post-meal glucose, segments most relevant to night time hypoglycemia risk, segments most relevant to post-meal hypoglycemia risk, segments most relevant to night time hyperglycemia risk, segments most relevant to post-meal hyperglycemia risk, segments most relevant to the determination of pre-meal insulin correction factor, and segments most relevant to the determination of post-meal insulin correction factor. The identified segments may be constructed from a single contiguous glucose history or multiple disparate glucose histories.

In certain embodiments, the value of glucose is stored for at least two glucose history segments. In certain embodiments, a first glucose history segment represents glucose values for a first time period; a second glucose history segment represents glucose values for a second time period, and so on. In certain embodiments, the value of an analyte is stored for a plurality of glucose history segments. In certain embodiments, the analyte history segments are not reset when insulin titration occurs.

In certain embodiments, the glucose value stored is related to basal glucose level. In certain embodiments, the glucose value stored is related to non-basal glucose levels.

In 902, the value of insulin doses associated with the glucose history segments of 901 is stored.

In some embodiments, the parameter is an insulin dose. In some embodiments, the parameter is a basal insulin dose. In some embodiments, the basal insulin dose changes across the plurality of analyte history segments. In some embodiments, the parameter is non-basal insulin dose. In some embodiments, the non-basal insulin dose does not change across the plurality of analyte history segments.

Examples of parameters include quantifiable values such as the dose amount of the long-acting insulin dose, and the fixed dose element of a mealtime rapid-acting (bolus) insulin dose.

Specific treatment choices, such as the specific choice of the long-acting insulin (e.g., degludec, glargine, detemir, icodec), the specific choice of the rapid-acting insulin (e.g., lispro, aspart, fiasp), the specific choice of a different type of insulin (e.g., pre-mixed, NPH), and the specific choice of other medication classes (e.g., biguanides, SGLT-2i, GLP-1Ra, DPP-4i, amylin analogues) can also be considered as treatment parameter by enumerating them.

For example, the choice of using or not using a particular treatment can be enumerated into 0 or 1, respectively. Furthermore, the choice can be enumerated into more than 2 values, corresponding to available dose options. For example, the choice of none, 0.25 mg, 0.5 mg, 1 mg, 1.7 mg, or 2.4 mg semaglutide dose can be enumerated into parameter values of 0, 1, 2, 3, 4, & 5 in that same order.

In 903 the data stored during method steps 901 and 902 are used to determine an adjustment value.

First, the value of glucose stored across a glucose history segment under method step 901 and the value of an insulin doses associated with the glucose history segments of 901 stored under method step 902 are correlated with each other. An exemplary correlation of these two values is shown in FIGS. 10A and 10B. Where the value of two parameter doses are equivalent, as shown in FIG. 11, the adjustment value is zero. Where the value of two parameter doses are non-equivalent, as shown in FIG. 12A, the adjustment value is non-zero.

Next, the value of the most recent insulin dose is identified. Then the value of a insulin dose not associated with the most recent analyte history segment is subtracted from the value of the most recent parameter dose. This equation is exemplified in FIG. 12B.

$$Δ {D}_{b} ( d - i ) = {D}_{b} ( d ) - {D}_{b} ( d - i )$$

wherein D_{b}(d) is the value of the most recent parameter dose, D_{b}(d−i) is the value of the parameter dose “i” analyte history segments before the most recent segment, and ΔD_{b}(d−i) is the difference between the two. This calculation yields the difference between the most recent parameter dose and a historic parameter dose.

Next, the value of the difference in the insulin dose is used to generate an adjustment value, wherein the adjustment value is the glucose value associated with an administration of a hypothetical insulin dose equivalent to the value of the difference previously calculated.

In some embodiments, the adjustment value can be determined according to the following method:
determining the hypothetical change in active insulin caused by a parameter dose of ΔD_{b}(d−i), as shown in FIG. 13A;
determining the hypothetical change in glucose concentration caused by the hypothetical change in active insulin of step 1, as shown in FIG. 13B; and
summing the individual hypothetical glucose concentration changes together, as shown in FIG. 13C.

As shown in FIGS. 13A-13D, when ΔD_{b}(d−i) is negative, the resulting change in active insulin is non-positive, and when ΔD_{b}(d−i) is positive, the resulting change in active insulin is non-negative. The amplitude and the temporal shape of the change in active insulin is a function of the dose difference ΔD_{b}(d−i) and specific pharmacokinetics/pharmacodynamics (PK/PD) of the particular insulin type. Note that the change in active insulin discussed here is an intermediary construct that is useful for the description to connect dose difference ΔD_{b}(d−i) with the hypothetical glucose concentration change.

In some embodiments, the hypothetical change in glucose concentration can be calculated with an insulin response model wherein an insulin sensitivity factor connects a particular insulin dose to a particular change in glucose expected when applying the insulin sensitivity factor to calculate a pre-meal correction dose.

In some embodiments, the patient's previous response to insulin is considered in calculating the adjustment value. In some embodiments, the patient's sensitivity to insulin is considered in calculating the adjustment value. In some embodiments, one or more of the patient's carbohydrate-to-insulin ratio, the patient's pre-meal correction factor, the patient's post-meal correction factor, the insulin action time, the desired dosing time of day for the long-acting (basal) insulin dose, the patient's state of health, the patient's level of physical activity, and the patient's meal consumption pattern is considered in calculating the adjustment value.

In 904 glucose values of the glucose history segments are adjusted to the values they would have been if the most recently administered insulin dose was administered during all the previous glucose history segments regardless of actual past insulin doses, based on the calculated adjustment value from method step 903. A graphical depiction of this adjustment being carried out is exemplified in FIG. 13D. This adjustment results in a continuous glucose history that is nearly identical to the hypothetical glucose history if all parameter doses were equivalent as shown by FIG. 14. In some embodiments, the analyte is basal glucose. In some embodiments, the parameter is basal insulin.

In 905 an updated insulin dose is generated based on the adjusted glucose history segments generated by method step 904. In some embodiments, the analyte is basal glucose. In some embodiments, the parameter is basal insulin. In some embodiments, there are additional parameters that are either set at a population level, determined at an individual level, or initialized at a population level and gradually individualized in generating the updated insulin dose.

In some embodiments, the insulin related parameter dose that is being titrated is a “parameter”. In some embodiments, the fixed basal insulin dose amount is a “parameter”. In some embodiments, the basal insulin dose is comprised of only the fixed dose element and the final dose administered is constant and equivalent to the fixed basal insulin dose parameter.

### Method for Determining Patient Insulin Sensitivity

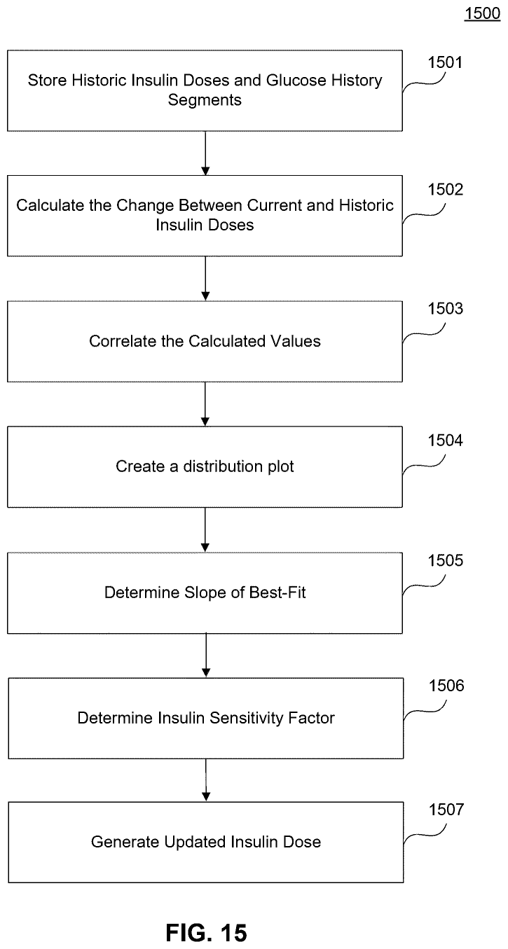

FIG. 15 is a flowchart illustrating a method 1500 to calculate a patient's insulin sensitivity, according to some embodiments. Method 1500 can be performed by processing logic that can comprise hardware (e.g., circuitry, dedicated logic, programmable logic, microcode, etc.), software (e.g., instructions executing on a processing device), or a combination thereof. It is to be appreciated that not all steps may be needed to perform the disclosure provided herein. Further, some of the steps may be performed simultaneously, or in a different order than shown in FIG. 15, as will be understood by a person of ordinary skill in the art(s).

In 1501, the value of glucose is stored across a glucose history segment. In some embodiments, this may be accomplished with a continuous analyte monitoring system. Examples of segments of analyte history include segments most relevant to fasting and/or night time, segments most relevant to pre-meal glucose, segments most relevant to post-meal glucose, segments most relevant to night time hypoglycemia risk, segments most relevant to post-meal hypoglycemia risk, segments most relevant to night time hyperglycemia risk, segments most relevant to post-meal hyperglycemia risk, segments most relevant to the determination of pre-meal insulin correction factor, and segments most relevant to the determination of post-meal insulin correction factor. The identified segments may be constructed from a single contiguous glucose history or multiple disparate glucose histories.

In certain embodiments, the value of glucose is stored for at least two glucose history segments. In certain embodiments, a first glucose history segment represents glucose values for a first time period; a second glucose history segment represents glucose values for a second time period, and so on. In certain embodiments, the value of an analyte is stored for a plurality of glucose history segments. In certain embodiments, the analyte history segments are not reset when insulin titration occurs.

In certain embodiments, the glucose value stored is related to basal glucose level. In certain embodiments, the glucose value stored is related to non-basal glucose levels.

In 1501 the value of insulin doses associated with the glucose history segments recorded in method step 1501 are also stored.

In some embodiments, the parameter is insulin. In some embodiments, the parameter is basal insulin. In some embodiments, the basal insulin dose changes across the plurality of analyte history segments. In some embodiments, the parameter is non-basal insulin. In some embodiments, the non-basal insulin dose does not change across the plurality of analyte history segments.

Examples of parameters include quantifiable values such as the dose amount of the long-acting insulin dose, and the fixed dose element of a mealtime rapid-acting (bolus) insulin dose.

Specific treatment choices, such as the specific choice of the long-acting insulin (e.g., degludec, glargine, detemir, icodec), the specific choice of the rapid-acting insulin (e.g., lispro, aspart, fiasp), the specific choice of a different type of insulin (e.g., pre-mixed, NPH), and the specific choice of other medication classes (e.g., biguanides, SGLT-2i, GLP-1Ra, DPP-4i, amylin analogues) can also be considered as treatment parameter by enumerating them.

For example, the choice of using or not using a particular treatment can be enumerated into 0 or 1, respectively. Furthermore, the choice can be enumerated into more than 2 values, corresponding to available dose options. For example, the choice of none, 0.25 mg, 0.5 mg, 1 mg, 1.7 mg, or 2.4 mg semaglutide dose can be enumerated into parameter values of 0, 1, 2, 3, 4, & 5 in that same order.

In 1502, the difference in the values between past and present insulin doses is calculated. The value of the most recent insulin dose is identified. Then the value of a insulin dose not associated with the most recent analyte history segment is subtracted from the value of the most recent parameter dose. This equation is exemplified in FIG. 12B.

$$Δ {D}_{b} ( d - i ) = {D}_{b} ( d ) - {D}_{b} ( d - i )$$

wherein D_{b}(d) is the value of the most recent parameter dose, D_{b}(d−i) is the value of the parameter dose “i” analyte history segments before the most recent segment, and ΔD_{b}(d−i) is the difference between the two. This calculation yields the difference between the most recent parameter dose and a historic parameter dose.

In 1503, the difference in the value of insulin doses generated by method step 1502 and the median S1 glucose derived from glucose history segment stored by method step 1501 are correlated. Specifically, the median S1 glucose of the glucose history segment associated with D_{b}(d−i) is correlated with the difference between insulin doses represented by ΔD_{b}(d−i).

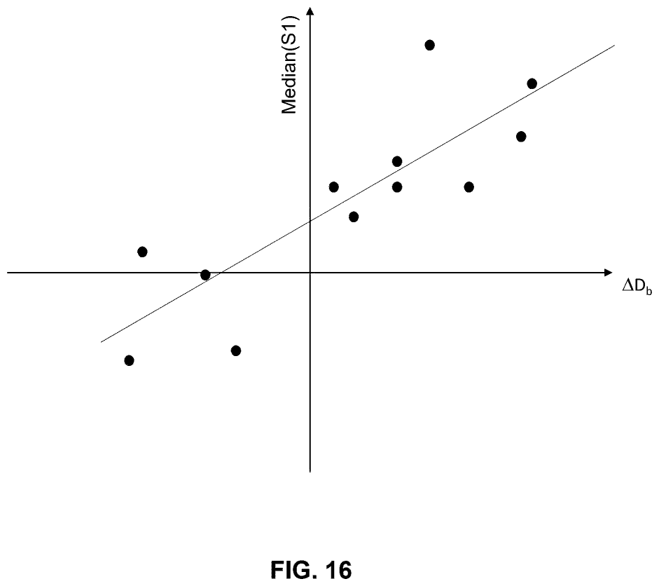

In 1504, the correlated values of method step 1503 are used to create a scatterplot (distribution plot) wherein the correlated values are charted so that the y-value is median S1 glucose and the x-value is ΔD_{b}(d−i). In some embodiments, two or more correlated values are charted as points to the scatterplot. In 1505 the scatterplot generated by method step 1504 is analyzed and a line of best fit is charted to the scatterplot. In some embodiments, the line of best fit is a linear regression. An exemplary scatterplot and best fit line generated by method steps 1504 and 1505 is shown in FIG. 16. In 1506 the slope of the best fit line generated by method step 1505 is determined and that slope is used to approximate the patient's insulin sensitivity. In addition, the y-intercept of the best fit line charted to the scatterplot may be used to define the patient's baseline insulin response.

In 1507, an updated insulin dose is generated based on the patient's insulin sensitivity factor and the adjusted glucose history segments generated by method step 904. In some embodiments, the patient's previous response to insulin is considered in generating the updated insulin dose. In some embodiments, one or more of the patient's carbohydrate-to-insulin ratio, the patient's pre-meal correction factor, the patient's post-meal correction factor, the insulin action time, the desired dosing time of day for the long-acting (basal) insulin dose, the patient's state of health, the patient's level of physical activity, and the patient's meal consumption pattern is considered in generating the updated insulin dose. In some embodiments, there are additional parameters that are either set at a population level, determined at an individual level, or initialized at a population level and gradually individualized in generating the updated insulin dose.

Based on the teachings contained in this disclosure, it will be apparent to persons skilled in the relevant art(s) how to employ the methods of this disclosure using data processing devices, computer systems and/or computer architectures other than those referenced in the present disclosure.

While this disclosure describes exemplary embodiments for exemplary fields and applications, it should be understood that the disclosure is not limited thereto. Other embodiments and modifications thereto are possible, and are within the scope and spirit of this disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments can perform functional blocks, steps, operations, methods, etc. using orderings different than those described herein.

References herein to “one embodiment,” “an embodiment,” “an example embodiment,” or similar phrases, indicate that the embodiment described can include a particular feature, structure, or characteristic, but every embodiment can not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein.

The breadth and scope of this disclosure should not be limited by any of the above-described exemplary embodiments, but should be defined only in accordance with the following claims and their equivalents.

Exemplary embodiments are set forth in the following numbered clauses:
1. A method for frequent titration of a parameter for generating recommended dosage amounts, the method comprising:
   storing a plurality of analyte history segments, wherein the plurality of analyte history segments further comprises a first segment representing a first set of analyte values for a first time period and a second segment representing a second set of analyte values for a second time period;
   storing a plurality of parameter doses associated with the plurality of analyte history segments, wherein the plurality of parameter doses each comprise a historic parameter dose;
   performing an analysis of the plurality of analyte history segments to determine a hypothetical adjustment value for the analyte values in the plurality of analyte history segments, wherein determining the hypothetical adjustment value comprises:
   generating a difference value by subtracting the value of the historic parameter dose from a most recent parameter dose; and setting the hypothetical adjustment value equal to a hypothetical change in analyte value caused by a hypothetical dose with the difference value;
   adjusting, based on the hypothetical adjustment value, the stored analyte value of each of the analyte history segments to generate an adjusted stored patient analyte value, wherein the adjusted stored patient analyte value represents the hypothetical patient analyte value associated with a hypothetical administration of a parameter dose equivalent to the most recent parameter dose; and
   generating an updated parameter dose based on the adjusted stored patient analyte value.
2. The method of clause 1, wherein the updated parameter dose is at a concentration which adjusts the adjusted stored patient analyte value to a homeostatic patient analyte value.
3. The method of clause 1, wherein one or more of: a patient's previous response to the parameter, a patient's sensitivity to the parameter, a patient's carbohydrate-to-parameter ratio, a patient's pre-meal correction factor, a patient's post-meal correction factor, a parameter action time, a desired dosing time of day for the long-acting parameter dose, a patient's state of health, a patient's level of physical activity, and a patient's meal consumption pattern are considered in calculating the adjustment value.
4. The method of clause 1, wherein the adjustment of the stored patient analyte value occurs at one or more fixed times of day, occurs when the requirements for another parameter dose titration is met, occurs dynamically upon a triggering event, or any combination thereof.
5. The method of clause 1, wherein the plurality of analyte history segments comprise all analyte history segments within a current rolling window, wherein the current rolling window includes a most recent parameter dose, an oldest parameter dose that affects the stored patient analyte values, and all intervening parameter doses occurring temporally between the two.
6. The method of clause 1, wherein the analyte history segments are not reset following titration of the parameter.
7. The method of clause 1, wherein two or more stored analyte values are adjusted.
8. The method of clause 1, wherein each of the stored analyte values in each analyte history segment is adjusted.
9. The method of clause 1, wherein the plurality of analyte history segments further comprises additional segments representing analyte values for additional time periods.
10. The method of clause 1, wherein the parameter is insulin.
11. The method of clause 10, wherein the stored insulin doses comprise at least one of a basal insulin dose or a non-basal insulin dose.
12. The method of clause 11, wherein the basal insulin dose changes across the plurality of analyte history segments.
13. The method of clause 11, wherein the non-basal insulin dose do not change across the plurality of analyte history segments.
14. The method of clause 12, further comprising calculating the change in basal insulin dose between a most recent administration and an administration from a previous analyte history segment; correlating the value of the change from the previous analyte history segment with the median adjusted analyte values for the previous analyte history segment; creating a distribution plot that charts the correlated values; determining a slope of a linear best-fit line charted to the distribution plot; determining an insulin sensitivity factor of the patient using the determined slope; and adjusting the insulin dose to be administered based on the insulin sensitivity factor of the patient.
15. The method of clause 12, further comprising:
   adjusting the insulin dose to be administered based on the insulin sensitivity factor of the patient.
16. A method for frequent titration of insulin for generating recommended dosage amounts, the method comprising:
   storing a plurality of glucose history segments, wherein the plurality of glucose history segments further comprises a first segment representing a first set of glucose values for a first time period and a second segment representing a second set of glucose values for a second time period;
   storing a plurality of insulin doses associated with the glucose history segments, wherein the plurality of insulin doses each comprise a historic insulin dose;
   performing an analysis of the plurality of glucose history segments to determine a hypothetical adjustment value for the glucose values in the plurality of glucose history segments, wherein determining the hypothetical adjustment value comprises:
   generating a difference value by subtracting the value of the historic insulin dose from a most recent insulin dose; and setting the hypothetical adjustment value equal to a hypothetical change in glucose value caused by a hypothetical dose with the difference value;
   adjusting, based on the hypothetical adjustment value, the stored glucose value of each of the analyte history segments to generate an adjusted stored patient glucose value, wherein the adjusted stored patient glucose value represents the hypothetical patient glucose value associated with a hypothetical administration of an insulin dose equivalent to the most recent insulin dose; and
   generating an updated insulin dose based on the adjusted stored patient glucose value.
17. The method of clause 16, wherein the updated insulin dose is at a concentration which adjusts the adjusted stored glucose analyte value to a homeostatic patient glucose value.
18. The method of clause 16, wherein one or more of: a patient's previous response to insulin, a patient's sensitivity to insulin, a patient's carbohydrate-to-insulin ratio, a patient's pre-meal insulin correction factor, a patient's post-meal insulin correction factor, an insulin's action time, a desired dosing time of day for the long-acting insulin dose, a patient's state of health, a patient's level of physical activity, and a patient's meal consumption pattern are considered in calculating the adjustment value.
19. The method of clause 16, wherein the adjustment of the stored patient glucose value occurs at one or more fixed times of day, occurs when the requirements for another insulin dose titration is met, occurs dynamically upon a triggering event, or any combination thereof.
20. The method of clause 16, wherein the plurality of glucose history segments comprise all glucose history segments within a current rolling window, wherein the current rolling window includes a most recent insulin dose, an oldest insulin dose that affects the stored patient analyte values, and all intervening insulin doses occurring temporally between the two.
21. The method of clause 16, wherein the glucose history segments are not reset following titration of insulin.
22. The method of clause 16, wherein the stored insulin doses comprise at least one of a basal insulin dose or a non-basal insulin doses.
23. The method of clause 22, wherein the basal insulin dose changes across the plurality of glucose history segments.
24. The method of clause 23, further comprising:
   calculating the change in basal insulin dose between a most recent administration and an administration from a previous analyte history segment;
   correlating the value of the change from the previous glucose history segment with the median adjusted glucose values for the previous glucose history segment; creating a distribution plot that charts the correlated values;
   determining a slope of a linear best-fit line charted to the distribution plot; determining an insulin sensitivity factor of the patient using the determined slope; and adjusting the insulin dose to be administered based on the insulin sensitivity factor of the patient.
25. The method of clause 23, further comprising:
   adjusting the insulin dose to be administered based on the insulin sensitivity factor of the patient.
26. A computing device in data communication with a skin-mounted assembly comprising an in vivo glucose sensor and a transmitter unit, the computing device comprising:
   one or more processors;
   one or more memory units operatively coupled to the one or more processors and including program instructions stored therein which, when executed by the one or more processors, causes the one or more processors to perform operations comprising:
   storing a plurality of analyte history segments, wherein the plurality of analyte history segments further comprises a first segment representing a first set of analyte values for a first time period and a second segment representing a second set of analyte values for a second time period;
   storing a plurality of parameter doses associated with the plurality of analyte history segments, wherein the plurality of parameter doses each comprise a historic parameter dose;
   performing an analysis of the plurality of analyte history segments to determine a hypothetical adjustment value for the analyte values in the plurality of analyte history segments, wherein determining the hypothetical adjustment value comprises:
   generating a difference value by subtracting the value of the historic parameter dose from a most recent parameter dose; and setting the hypothetical adjustment value equal to a hypothetical change in analyte value caused by a hypothetical dose with the difference value;
   adjusting, based on the hypothetical adjustment value, the stored patient analyte value of each of the analyte history segments to generate an adjusted stored patient analyte value, wherein the adjusted stored patient analyte value represents the hypothetical patient analyte value associated with a hypothetical administration of a parameter dose equivalent to the most recent parameter dose; and
   generating an updated parameter dose based on the adjusted analyte value.
27. The computing device of clause 26, wherein the updated parameter dose is at a concentration which adjusts the adjusted stored patient analyte value to a homeostatic patient analyte value.
28. The computing device of clause 26, wherein one or more of: a patient's previous response to the parameter, a patient's sensitivity to the parameter, a patient's carbohydrate-to-parameter ratio, a patient's pre-meal correction factor, a patient's post-meal correction factor, a parameter action time, a desired dosing time of day for the long-acting parameter dose, a patient's state of health, a patient's level of physical activity, and the patient's meal consumption pattern are considered in calculating the adjustment value.
29. The computing device of clause 26 wherein the adjustment of the stored patient analyte value occurs at one or more fixed times of day, occurs when the requirements for another parameter dose titration is met, occurs dynamically upon a triggering event, or any combination thereof.
30. The computing device of clause 26, wherein the plurality of analyte history segments comprise all analyte history segments within a current rolling window, wherein the current rolling window includes a most recent parameter dose, an oldest parameter dose that affects the stored patient analyte values, and all intervening parameter doses occurring temporally between the two.
31. The computing device of clause 26, wherein the analyte history segments are not reset following titration of the parameter.
32. The computing device of clause 26, wherein the parameter is insulin.
33. The computing device of clause 32, wherein the stored insulin doses comprise at least one of a basal insulin dose or a non-basal insulin doses.
34. The computing device of clause 32, wherein the insulin dose changes across the plurality of analyte history segments.
35. The computing device of clause 34, wherein the operations further comprise:
   calculating the change in basal insulin dose between a most recent administration and an administration from a previous analyte history segment;
   correlating the value of the change from the previous glucose history segment with the median adjusted glucose values for the previous glucose history segment;
   creating a distribution plot that charts the correlated values;
   determining a slope of a linear best-fit line charted to the distribution plot;
   determining a insulin sensitivity factor of the patient using the determined slope; and
   adjusting the insulin dose to be administered based on the insulin sensitivity factor of the patient.
36. The computing device of clause 34, wherein the operations further comprise:
   adjusting the insulin dose to be administered based on the insulin sensitivity factor of the patient.

What is claimed is:

1. A method for frequent titration of a parameter for generating recommended dosage amounts, the method comprising:
storing a plurality of analyte history segments, wherein the plurality of analyte history segments further comprises a first segment representing a first set of analyte values for a first time period and a second segment representing a second set of analyte values for a second time period;
storing a plurality of parameter doses associated with the plurality of analyte history segments, wherein the plurality of parameter doses each comprise a historic parameter dose;
performing an analysis of the plurality of analyte history segments to determine a hypothetical adjustment value for the analyte values in the plurality of analyte history segments, wherein determining the hypothetical adjustment value comprises:
generating a difference value by subtracting a historic parameter dose value from a most recent parameter dose value; and
setting the hypothetical adjustment value equal to a hypothetical change in analyte value caused by a hypothetical dose with the difference value;
adjusting, based on the hypothetical adjustment value, a stored analyte value of each of the plurality of analyte history segments to generate an adjusted stored patient analyte value, wherein the adjusted stored patient analyte value represents the hypothetical patient analyte value associated with a hypothetical administration of a parameter dose equivalent to the most recent parameter dose; and
generating an updated parameter dose based on the adjusted stored patient analyte value.

2. The method of claim 1, wherein the analyte history segments are not reset following titration of the parameter.

3. The method of claim 1, wherein two or more stored analyte values are adjusted.

4. The method of claim 1, wherein the plurality of analyte history segments further comprises additional segments representing analyte values for additional time periods.

5. The method of claim 1, wherein the parameter comprises stored insulin doses.

6. The method of claim 5, wherein the stored insulin doses comprise at least one of a basal insulin dose or a non-basal insulin dose.

7. The method of claim 6, wherein the basal insulin dose changes across the plurality of analyte history segments.

8. The method of claim 6, wherein the non-basal insulin dose do not change across the plurality of analyte history segments.

9. The method of claim 7, further comprising:
calculating the change in basal insulin dose between a most recent administration and an administration from a previous analyte history segment;
correlating a value of the change from the previous analyte history segment with median adjusted analyte values for the previous analyte history segment;
creating a distribution plot that charts the correlated values;
determining a slope of a linear best-fit line charted to the distribution plot;
determining an insulin sensitivity factor of the patient using the determined slope; and
adjusting an insulin dose to be administered based on the insulin sensitivity factor of the patient.

10. A method for frequent titration of insulin for generating recommended dosage amounts based on a parameter, the method comprising:
storing a plurality of glucose history segments, wherein the plurality of glucose history segments further comprises a first segment representing a first set of glucose values for a first time period and a second segment representing a second set of glucose values for a second time period;
storing a plurality of insulin doses associated with the plurality of glucose history segments, wherein the plurality of insulin doses each comprise a historic insulin dose;
performing an analysis of the plurality of glucose history segments to determine a hypothetical adjustment value for the glucose values in the plurality of glucose history segments, wherein determining the hypothetical adjustment value comprises:
generating a difference value by subtracting a historic parameter dose value from a most recent parameter dose value; and
setting the hypothetical adjustment value equal to a hypothetical change in analyte value caused by a hypothetical dose with the difference value;
adjusting, based on the hypothetical adjustment value, a stored analyte value of each of the plurality of analyte history segments to generate an adjusted stored patient analyte value, wherein the adjusted stored patient analyte value represents the hypothetical patient analyte value associated with a hypothetical administration of a parameter dose equivalent to the most recent parameter dose; and
generating an updated insulin dose based on the adjusted stored patient glucose value.

11. The method of claim 10, wherein the glucose history segments are not reset following titration of insulin.

12. The method of claim 10, wherein the parameter comprises stored insulin doses and wherein the stored insulin doses comprise at least one of a basal insulin dose or a non-basal insulin doses.

13. The method of claim 12, wherein the basal insulin dose changes across the plurality of glucose history segments.

14. The method of claim 13, further comprising:
calculating the change in basal insulin dose between a most recent administration and an administration from a previous analyte history segment;
correlating a value of the change from a previous glucose history segment with median adjusted glucose values for the previous glucose history segment;
creating a distribution plot that charts the correlated values;
determining a slope of a linear best-fit line charted to the distribution plot;
determining a insulin sensitivity factor of the patient using the determined slope; and
adjusting the insulin dose to be administered based on the insulin sensitivity factor of the patient.

15. A computing device in data communication with a skin-mounted assembly comprising an in vivo glucose sensor and a transmitter unit, the computing device comprising:
one or more processors;
one or more memory units operatively coupled to the one or more processors and including program instructions stored therein which, when executed by the one or more processors, causes the one or more processors to perform operations comprising:
storing a plurality of analyte history segments, wherein the plurality of analyte history segments further comprises a first segment representing a first set of analyte values for a first time period and a second segment representing a second set of analyte values for a second time period;
storing a plurality of parameter doses associated with the plurality of analyte history segments, wherein the plurality of parameter doses each comprise a historic parameter dose, and wherein the plurality of parameter doses is associated with a parameter;
performing an analysis of the plurality of analyte history segments to determine a hypothetical adjustment value for the analyte values in the plurality of analyte history segments, wherein determining the hypothetical adjustment value comprises:
generating a difference value by subtracting a historic parameter dose value from a most recent parameter dose value; and
setting the hypothetical adjustment value equal to a hypothetical change in analyte value caused by a hypothetical dose with the difference value;
adjusting, based on the hypothetical adjustment value, a stored analyte value of each of the plurality of analyte history segments to generate an adjusted stored patient analyte value, wherein the adjusted stored patient analyte value represents the hypothetical patient analyte value associated with a hypothetical administration of a parameter dose equivalent to the most recent parameter dose; and
generating an updated parameter dose based on the adjusted analyte value.

16. The computing device of claim 15, wherein the analyte history segments are not reset following titration of the parameter.

17. The computing device of claim 15, wherein the parameter comprises stored insulin doses.

18. The computing device of claim 17, wherein the stored insulin doses comprise at least one of a basal insulin dose or a non-basal insulin doses.

19. The computing device of claim 17, wherein the insulin dose changes across the plurality of analyte history segments.

20. The computing device of claim 19, wherein the operations further comprise:
calculating the change in basal insulin dose between a most recent administration and an administration from a previous analyte history segment;
correlating a value of the change from a previous glucose history segment with median adjusted glucose values for the previous glucose history segment;
creating a distribution plot that charts the correlated values;
determining a slope of a linear best-fit line charted to the distribution plot;
determining a insulin sensitivity factor of the patient using the determined slope; and
adjusting the insulin dose to be administered based on the insulin sensitivity factor of the patient.
